(19) United States
(12) Patent Application Publication
(10) Pub. No.: US 2025/0230321 A1
(43) Pub. Date: Jul. 17, 2025

(54) SEMICONDUCTOR DEVICE PROCESSING COMPOSITION, COMPOUND, METHOD FOR PRODUCING MODIFIED SUBSTRATE, AND METHOD FOR PRODUCING SEMICONDUCTOR DEVICE

(71) Applicant: FUJIFILM CORPORATION, Tokyo (JP)

(72) Inventors: Naoya Shimoju, Shizuoka (JP); Kota Muramatsu, Shizuoka (JP); Katsuya Kondo, Shizuoka (JP); Akihiro Hakamata, Shizuoka (JP); Tomonori Takahashi, Shizuoka (JP); Atsushi Mizutani, Shizuoka (JP); Koichi Sato, Shizuoka (JP)

(21) Appl. No.: 19/097,110

(22) Filed: Apr. 1, 2025

Related U.S. Application Data
(63) Continuation of application No. PCT/JP2023/039693, filed on Nov. 2, 2023.

(30) Foreign Application Priority Data
Nov. 17, 2022 (JP) 2022-184298
Feb. 16, 2023 (JP) 2023-022529
Sep. 19, 2023 (JP) 2023-151072

(51) Int. Cl. C09D 4/00; C07C 217/72; C07F 9/38; C09D 125/18; C09D 129/10; C09D 133/14; C09D 133/26; C09D 135/00; C09D 171/00; C23C 16/04; C23C 16/34; C23C 16/40; C23C 16/455; H01L 21/02

(52) U.S. Cl. CPC C09D 4/00; C07C 217/72; C07F 9/38; C09D 125/18; C09D 129/10; C09D 133/14; C09D 133/26; C09D 135/00; C09D 171/00; C23C 16/04; C23C 16/34; C23C 16/403; C23C 16/45525; H01L 21/02118; H01L 21/0228

(57) ABSTRACT

The present invention provides a composition for treating a semiconductor device, a compound, a method for producing a modified substrate, and a method for producing a semiconductor device, each of which can form a coating film having a high coat-forming property for inhibiting the formation of an ALD coating film. The composition for treating a semiconductor device of an embodiment of the present invention includes a compound having a specific functional group bonded to or adsorbed on a substrate and a polymerizable group, and a solvent, in which the specific functional group is a basic functional group or an acidic functional group, in a case where the specific functional group is the basic functional group, an acid dissociation constant of a conjugate acid of a compound obtained by adding a proton to the basic functional group is 7.0 or more, and in a case where the specific functional group is the acidic functional group, an acid dissociation constant of the compound upon dissociation of a proton from the acidic functional group is 5.0 or less.

### CROSS-REFERENCE TO RELATED APPLICATIONS

This application is a Continuation of PCT International Application No. PCT/JP2023/039693 filed on Nov. 2, 2023, which claims priority under 35 U.S.C. § 119(a) to Japanese Patent Application No. 2022-184298 filed on Nov. 17, 2022, Japanese Patent Application No. 2023-022529 filed on Feb. 16, 2023 and Japanese Patent Application No. 2023-151072 filed on Sep. 19, 2023. The above applications are hereby expressly incorporated by reference, in their entirety, into the present application.

### BACKGROUND OF THE INVENTION

### 1. Field of the Invention

The present invention relates to a composition for treating a semiconductor device, a compound, a method for producing a modified substrate, and a method for producing a semiconductor device.

### 2. Description of the Related Art

With the further miniaturization of semiconductor devices, there is a demand for the formation of finer and more precise semiconductor elements. In the related art, photolithography has been used in the formation of a semiconductor element, but it requires registration of patterns or the like and is no longer capable of meeting the accuracy required these days.

Currently, atomic layer deposition (ALD) is known as a technique for forming a film in a predetermined region. As a related technique using such the ALD, for example, JP2020-107855A describes “a substrate treatment method in which monomers are polymerized on a substrate to form a polymer film, the method including a step of supplying, onto the substrate, the monomers that chemically bond with the substrate, and a step of supplying, onto the substrate to which the monomers have been supplied, an initiator that polymerizes the monomers,” as a substrate treatment method that can accomplish at least one of objects of selectively forming a film on a substrate and suppressing the residual metal after the film formation.

### SUMMARY OF THE INVENTION

On the other hand, in recent years, studies have been conducted on a method for forming a film (modified film) by modifying one region of a substrate having a plurality of regions (for example, a metal region including metal atoms and an insulating region including an insulator) consisting of different materials on the surface, and then performing an ALD treatment, whereby a coating film with ALD (ALD coating film) is not formed in a region where the modified film is not formed, and the ALD coating film is formed in a region where the modified film is formed, thereby forming a fine pattern.

With reference to the substrate treatment method described in JP2020-107855A, the present inventors have made attempted to form a film of a polymer as the modified film, and then to form an ALD coating film on a region where the modified film was not formed. However, as a result, a thick ALD coating film was formed even on a region where the modified film was formed (on the modified film). That is, it has been clarified that in a film of the polymer obtained by using the monomers used in the substrate treatment method described in JP2020-107855A, the formation of the ALD coating film cannot be sufficiently inhibited and further improvement is required.

Therefore, an object of the present invention is to provide a composition for treating a semiconductor device, the composition making it possible to form a coating film having high inhibitory properties for formation of an ALD coating film.

In addition, another object of the present invention is to provide a compound, a method for producing a modified substrate, and a method for producing a semiconductor device.

The present inventors have conducted extensive studies to accomplish the object, and as a result, have completed the present invention. That is, the present inventors have found that the object can be accomplished by the following configurations.
[1] A composition for treating a semiconductor device, the composition comprising:
   a compound having a specific functional group bonded to or adsorbed on a substrate and a polymerizable group; and
   a solvent,
   in which the specific functional group is a basic functional group or an acidic functional group,
   in a case where the specific functional group is the basic functional group, an acid dissociation constant of a conjugate acid of a compound obtained by adding a proton to the basic functional group is 7.0 or more, and
   in a case where the specific functional group is the acidic functional group, an acid dissociation constant of the compound upon dissociation of a proton from the acidic functional group is 5.0 or less.
[2] The composition for treating a semiconductor device according to [1],
   in which the basic functional group is an amino group, a hydrazine group, or a guanidine group.
[3] The composition for treating a semiconductor device according to [1] or [2],
   in which the basic functional group is a primary amino group, a secondary amino group, or a tertiary amino group.
[4] The composition for treating a semiconductor device according to any one of [1] to [3],
   in which the basic functional group is a primary amino group.
[5] The composition for treating a semiconductor device according to any one of [1] to [4],
   in which the acidic functional group is a phosphonic acid group, a sulfo group, or a carboxy group.
[6] The composition for treating a semiconductor device according to any one of [1] to [5],
   in which the acidic functional group is a phosphonic acid group or a sulfo group.
[7] The composition for treating a semiconductor device according to any one of [1] to [6],
   in which the polymerizable group is an ethylenically unsaturated group.
[8] The composition for treating a semiconductor device according to any one of [1] to [7],
   in which the polymerizable group is an aromatic vinyl group, an acryloyloxy group, a methacryloyloxy group, an acrylamide group, a methacrylamide group, a maleimide group, or a vinyl ether group.
[9] The composition for treating a semiconductor device according to any one of [1] to [8],
   in which the polymerizable group is a styryl group or a vinylnaphthyl group.
[10] The composition for treating a semiconductor device according to any one of [1] to [9],
   in which the compound is a compound represented by General Formula (1) which will be described later.
[11] The composition for treating a semiconductor device according to any one of [1] to [9],
   in which the compound is a compound represented by General Formula (4) which will be described later.
[12] The composition for treating a semiconductor device according to any one of [1] to [11],
   in which a content of the compound is 3.00% by mass or less with respect to a total mass of the composition for treating a semiconductor device.
[13] The composition for treating a semiconductor device according to any one of [1] to [12],
   in which a total amount of the compound and the solvent is 99.90% by mass or more with respect to a total mass of the composition for treating a semiconductor device.
[14] The composition for treating a semiconductor device according to any one of [1] to [13],
   in which a water contact angle of a film obtained by applying the composition for treating a semiconductor device is 60 degrees or more.
[15] A compound represented by General Formula (2) which will be described later.
[16] A compound represented by General Formula (3) which will be described later.
[17] A compound represented by General Formula (5) which will be described later.
[18] A method for producing a modified substrate, the method comprising:
   a step 1 of bringing a substrate having at least two surfaces of a first surface and a second surface, which are composed of materials different from each other, into contact with the composition for treating a semiconductor device according to any one of [1] to [14], to form a first film on the first surface; and
   a step 2 of subjecting the substrate obtained in the step 1 to an atomic layer deposition treatment (ALD treatment) to form a second film on the second surface.
[19] A method for producing a semiconductor device, the method comprising:
   the method for producing a modified substrate according to [18].

According to the present invention, it is possible to provide a composition for treating a semiconductor device, which is capable of forming a coating film having high inhibitory properties for formation of an ALD coating film.

In addition, according to the present invention, it is possible to provide a compound, a method for producing a modified substrate, and a method for producing a semiconductor device.

### DESCRIPTION OF THE PREFERRED EMBODIMENTS

Hereinafter, the present invention will be described in detail.

Description of configuration requirements described below may be made on the basis of representative embodiments of the present invention in some cases, but the present invention is not limited to such embodiments.

Hereinafter, the same meanings are used in the present specification.

In the present specification, a numerical value range expressed using “to” means a range that includes the preceding and succeeding numerical values of “to” as the lower limit value and the upper limit value, respectively.

In the present specification, the “total solid content” means the total content of all components contained in the composition other than a solvent such as water or an organic solvent.

A compound described in the present specification may include a structural isomer, an optical isomer, and an isotope unless otherwise specified. In addition, one kind of structural isomer, optical isomer, and isotope may be included, or two or more kinds thereof may be included.

In the present specification, with regard to the bonding direction of a divalent group (for example, —COO—), unless otherwise specified, in a case where Y in a compound represented by “X—Y—Z” is —COO—, the compound may be either “X—O—CO—Z” or “X—CO—O—Z”.

In the present specification, pKa is a value determined by computation from a value based on a Hammett substituent constant and the database of known literature values, using the following software package 1.

Software Package 1: Advanced Chemistry Development (ACD/Labs) Software V 8.14 for Solaris (1994-2007 ACD/Labs)

Furthermore, in a case where the pKa cannot be calculated by the method, a value determined by a molecular orbital calculation method is adopted. As a specific method using the molecular orbital calculation method, a value determined by using Gaussian 16 based on DFT is adopted.

In the present specification, unless otherwise specified, a molecular weight of a compound having a molecular weight distribution is a weight-average molecular weight.

Hereinafter, a composition for treating a semiconductor device of an embodiment of the present invention (hereinafter also simply referred to as “the present composition”) will be described in detail.

The present composition is a composition for treating a semiconductor device, the composition including a compound having a specific functional group bonded to or adsorbed on a substrate and a polymerizable group, and a solvent, in which the specific functional group is a basic functional group or an acidic functional group, in a case where the specific functional group is the basic functional group, an acid dissociation constant of a conjugate acid of a compound obtained by adding a proton to the basic functional group is 7.0 or more, and in a case where the specific functional group is the acidic functional group, an acid dissociation constant of the compound upon dissociation of a proton from the acidic functional group is 5.0 or less.

The mechanism by which the object of the present invention can be accomplished by adopting the configurations of the present invention is not necessarily clear, but is presumed to be as follows by the present inventors.

Furthermore, the mechanism by which the effect is obtained is not limited by the following supposition. In other words, even in a case where an effect is obtained by a mechanism other than the following, it is included in the scope of the present invention.

Since the acid dissociation constant of the conjugate acid of the specific compound among the specific compounds included in the present composition, or the acid dissociation constant of the specific compound is within a predetermined range, the specific functional group can be selectively bonded to or adsorbed on a specific region on a substrate, and a film consisting of the specific compound can be formed region-selectively.

Further, since the specific compound has a polymerizable group, the substrate is heated in the ALD treatment, whereby the polymerizable groups react with each other to form a covalent bond and the film is cured to obtain a coating film that is a cured film.

Therefore, it is presumed that a film is formed of the present composition in a region on a substrate where the formation of a coating film by ALD is to be inhibited, and the polymerizable groups are covalently bonded to each other in the ALD treatment, whereby a coating film (cured film) having excellent heat resistance and water repellency is formed, and therefore, the ALD coating film is hardly formed on the cured film in the ALD treatment.

Hereinafter, components that can be included in the composition for treating a semiconductor device of the embodiment of the present invention will be described in detail.

Furthermore, the fact that a coating film having higher inhibitory properties for formation of an ALD coating film can be formed by the present composition is also referred to as “the effect of the present invention being more excellent”.

### Composition for Treating Semiconductor Device

The present composition (composition for treating a semiconductor device) includes a compound (specific compound) having a specific functional group bonded to or adsorbed on a substrate and a polymerizable group, and a solvent.

### (Specific Compound)

As described above, the specific compound is a compound having a specific functional group and a polymerizable group. The bonding position of each group is not particularly limited, but it is preferable that the polymerizable group has a specific functional group at one end part of the molecule and has a polymerizable group at the other end part of the molecule.

The specific functional group is a functional group bonded to or adsorbed on a substrate, but may form any bond or interaction such as a covalent bond, a coordinate bond, an ionic bond, a hydrogen bond, a Van der Waals bond, and a metal bond with the substrate.

As the specific functional group, a functional group bonded to or adsorbed on a surface including a metal atom is preferable, and a functional group bonded to or adsorbed on a metal surface is more preferable.

Examples of the metal include transition metals, and copper, cobalt, titanium, tantalum, tungsten, ruthenium, or molybdenum is preferable.

In a case where the specific functional group is a basic functional group, an acid dissociation constant (hereinafter also referred to as a “pKa (b)”) of a conjugate acid of the specific compound obtained by adding a proton to the basic functional group is 7.0 or more.

For example, in a case where the specific compound is the following compound E-3, an equilibrium state for the pKa (b) is represented by the following dissociation equilibrium expression, and the pKa (b) is 8.2.

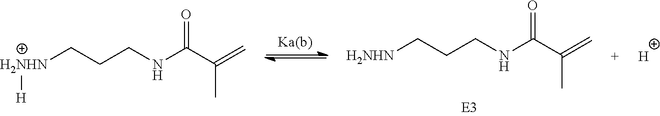

In a case where the pKa (b) is 7.0 or more, the specific functional group is easily adsorbed on or bonded to a substrate (in particular, a substrate having a tungsten surface or a ruthenium surface), and a coating film exhibiting predetermined characteristics is easily and stably formed on the substrate.

The pKa (b) is preferably 8.0 or more, more preferably 9.0 or more, and still more preferably 10.0 or more. The upper limit is not particularly limited, but is preferably 30.0 or less.

Examples of the basic functional group include nitrogen-containing groups, and among these, the basic functional group is preferably an amino group, a hydrazine group, or a guanidine group, more preferably a primary amino group, a secondary amino group, or a tertiary amino group, and still more preferably the primary amino group.

Furthermore, in the present specification, an amino group included in a hydrazine group or a guanidine group is not included in the amino group (the primary amino group, the secondary amino group, or the tertiary amino group). That is, the hydrazine group and the guanidine group in the present specification are treated as groups different from the amino group.

The nitrogen-containing group may be a group obtained by removing one hydrogen atom from an alicyclic amine. Examples of the alicyclic amine include pyrrolidine, piperidine, morpholine, 1,4-diazabicyclo [2.2.2]octane (DABCO (registered trademark)), 1,8-diazabicyclo[5.4.0]-7-undecene (DBU (registered trademark)), 7-methyl-1,5,7-triazabicyclo[4.4.0]dec-5-ene, and 1,5,7-triazabicyclo [4.4.0]dec-5-ene.

In addition to the above, examples of the nitrogen-containing group also include a group obtained by removing one hydrogen atom from 2-methylimidazole.

The basic functional group is preferably a group represented by any of Formulae (S1) to (S3). In Formulae (S1) to (S3), * represents a bonding position.

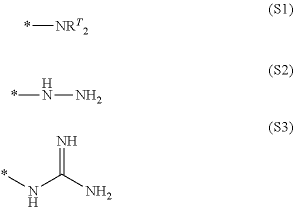

In Formula (S1), R^{T}'s each independently represent a hydrogen atom or an alkyl group having 1 to 3 carbon atoms.

A plurality of R^{T}'s may be bonded to each other to form a ring. The ring to be formed is a ring including a nitrogen atom, and examples thereof include a pyrrolidine ring, a piperidine ring, and a piperazine ring.

In a case where the specific functional group is an acidic functional group, an acid dissociation constant (hereinafter also referred to as a “pKa (a)”) of the specific compound in a case where a proton is dissociated from the acidic functional group is 5.0 or less.

For example, in a case where the specific compound is the following compound E-15, the equilibrium state for the pKa (a) is represented by the following dissociation equilibrium expression, and the pKa (a) is 4.8.

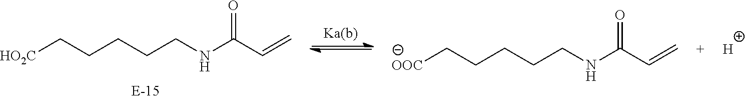

In a case where the pKa (a) is 5.0 or less, the specific functional group is easily adsorbed on or bonded to a substrate (in particular, a substrate having a copper surface), and a coating film exhibiting predetermined characteristics is easily and stably formed on the substrate.

The pKa (a) is preferably 4.0 or less, more preferably 3.5 or less, and still more preferably 3.0 or less. The lower limit is not particularly limited, but is preferably −5.0 or more.

Examples of the acidic functional group include a phosphoric acid group (—PO₄H₂), a phosphonic acid group (—PO₃H₂), a sulfo group (—SO₃H), a carboxy group (—COOH), and salts thereof. As the acidic functional group, the phosphonic acid group, the sulfo group, or the carboxy group is preferable, and the phosphonic acid group or the sulfo group is more preferable.

The salt of the phosphoric acid group refers to a group represented by —PO₄²⁻Ctⁿ⁺_{2/n}. Furthermore, Ctⁿ⁺ represents an n-valent cation and n represents 1 or 2. Examples of the monovalent cation include Li⁺, Na⁺, K⁺, and NH₄⁺. In a case where Ctⁿ⁺ represents a monovalent cation, the number thereof is 2. Examples of the divalent cation include Mg²⁺ and Ca²⁺. In a case where Ctⁿ⁺ represents a divalent cation, the number thereof is 1.

Furthermore, the compound having a phosphoric acid group is also referred to as a “phosphoric compound” and the functional group name is also referred to as “-phosphoric acid”.

The salt of the phosphonic acid group refers to a group represented by —PO₃²⁻Ctⁿ⁺_{2/n}. Ctⁿ⁺ represents an n-valent cation, where n represents 1 or 2. Examples of the monovalent cation and the divalent cation include the same cations as the cations described in the salt of the phosphoric acid group above, and the numbers thereof are also the same.

Furthermore, the compound having a phosphonic acid group is also referred to as a “phosphonic acid compound”.

The salt of the sulfo group refers to a group represented by —SO₃⁻Ct⁺. Ct⁺ represents a monovalent cation, and examples thereof include the same cations as the monovalent cations described in the salt of the phosphoric acid group.

The salt of the carboxy group refers to a group represented by —COO⁻Ct⁺. Ct⁺ represents a monovalent cation, and examples thereof include the same cations as the monovalent cations described in the salt of the phosphoric acid group.

The number of the specific functional groups contained in the specific compound is not particularly limited as long as it is 1 or more, but the number of the specific functional groups is preferably 1 to 3, and more preferably 1 or 2.

The polymerizable group is not particularly limited, but is preferably a polymerizable group in which a polymerization reaction proceeds by heating. After forming a film consisting of the specific compound on a substrate, the polymerizable groups are reacted with each other by a heat treatment, whereby the film consisting of the specific compound is a coating film having excellent heat resistance and high inhibitory properties for formation of an ALD coating film. In addition, since the coating film has excellent heat resistance, it is possible to widen the process window in a semiconductor device treating step.

Examples of the polymerizable group include a radically polymerizable group, a cationically polymerizable group, and an anionically polymerizable group, and an ethylenically unsaturated group is preferable. The ethylenically unsaturated group is a functional group having an ethylenically unsaturated bond. Among these, as the polymerizable group, an aromatic vinyl group, an acryloyloxy group (CH₂═CH—COO—), a methacryloyloxy group (CH₂═CCH₃—COO—), an acrylamide group (CH₂═CH—CONH—), a methacrylamide group (CH₂═CCH₃—CONH—), a maleimide group, a vinyl group, or a vinyl ether group is preferable, and a styryl group or a vinylnaphthyl group is more preferable.

The aromatic vinyl group refers to a group obtained by substituting a hydrogen atom on an aryl group or a heteroaryl group with a vinyl group. Among these, a styryl group or a vinylnaphthyl group is preferable as the aromatic vinyl group.

Furthermore, the vinyl naphthyl group is preferably a group obtained by removing a hydrogen atom at the 6-position from 2-vinylnaphthalene.

In addition, in the present specification, each of the groups exemplified above as the polymerizable group is treated as a group different from the vinyl group (CH₂═CH—). That is, each group exemplified above as the polymerizable group includes a structure represented by CH₂═CH—, but is treated as a group different from the vinyl group.

The number of polymerizable groups contained in the specific compound is not particularly limited as long as it is 1 or more, and is preferably 1 to 3, and more preferably 1 or 2.

The specific compound preferably has a structure exhibiting alignment properties. The structure exhibiting alignment properties refers to a structure having a function of aligning the specific compound in a direction perpendicular to the substrate in a case where the substrate and the present composition are brought into contact with each other to form a film consisting of the specific compound on the substrate.

The structure exhibiting alignment properties is not particularly limited, and examples thereof include a divalent aliphatic hydrocarbon group which may have an etheric oxygen atom or —CO—, a divalent aromatic ring group, and a group formed by a combination thereof. Among these, the divalent aliphatic hydrocarbon group which may have an etheric oxygen atom is preferable.

The divalent aliphatic hydrocarbon group may be linear, branched, or cyclic, but is preferably linear. Examples of the divalent aliphatic hydrocarbon group include an alkylene group, an alkenylene group, and an alkynylene group, and the alkylene group is preferable.

The number of carbon atoms in the divalent aliphatic hydrocarbon group is not particularly limited, but from the viewpoint of improving the stability of the film consisting of the specific compound on the substrate, the number of carbon atoms is preferably 6 to 30, more preferably 8 to 28, and still more preferably 10 to 24.

The divalent aromatic ring group may be any of a divalent aromatic hydrocarbon group (arylene group) or a divalent aromatic heterocyclic group (heteroarylene group), but is preferably an arylene group.

The divalent aromatic ring group may be a monocycle or a polycycle.

The number of carbon atoms in the divalent aromatic ring group is preferably 5 to 25, more preferably 6 to 20, and still more preferably 6 to 10.

Examples of the arylene group include a phenylene group. In addition, the arylene group may have a structure in which a plurality of aromatic hydrocarbon rings are bonded through a single bond, such as a biphenyl structure.

Examples of the heteroarylene group include a group obtained by removing two hydrogen atoms from pyridine.

The specific compound is preferably a compound represented by General Formula (S1), and more preferably a compound represented by General Formula (1).

X¹-L¹-Y¹ General Formula (S1)

In General Formula (S1), X¹ represents an amino group, a hydrazine group, a guanidine group, a phosphonic acid group, a sulfo group, or a carboxy group.

Specific aspects and suitable aspects of each group represented by X¹ are as described above for the specific functional group. Among these, a primary amino group is preferable as each group represented by X¹.

In General Formula (S1), Y¹ represents an ethylenically unsaturated group.

As the ethylenically unsaturated group represented by Y¹, a styryl group, a vinylnaphthyl group, an acryloyloxy group, a methacryloyloxy group, an acrylamide group, a methacrylamide group, a maleimide group, a vinyl group, or a vinyl ether group is preferable.

In General Formula (S1), L¹ represents an etheric oxygen atom, a divalent aliphatic hydrocarbon group which may have —CO—, a divalent aromatic ring group, or a group formed by a combination of these.

Specific aspects and suitable aspects of the divalent aliphatic hydrocarbon group, the divalent aromatic ring group, and the group formed by a combination thereof are as described above.

Among these, the divalent aliphatic hydrocarbon group is preferably an alkylene group which may have an etheric oxygen atom. The number of carbon atoms in the alkylene group is preferably 1 to 30.

As the divalent aromatic ring group, a phenylene group is preferable.

Among these, the specific compound is preferably the compound represented by General Formula (1).

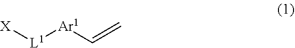

In General Formula (1),
X represents a primary amino group, a phosphonic acid group, or a sulfo group.
L¹ represents a single bond or an alkylene group which may have an etheric oxygen atom.
Ar¹ represents an arylene group.

The alkylene group which may have an etheric oxygen atom, represented by L¹, may be linear, branched, or cyclic, but is preferably linear.

In addition, the number of carbon atoms in the alkylene group which may have an etheric oxygen atom is preferably 6 to 30, more preferably 8 to 28, and still more preferably 10 to 24.

The alkylene group which may have an etheric oxygen atom, represented by L¹, is preferably a group represented by Formula (1a) or Formula (1b).

*-L^{1a}-O—** Formula (1a)

*-L^{1b}-O—CH₂—* Formula (1b)

In Formulae (1a) and (1b), * represents a bonding position with X in General Formula (1), and ** represents a bonding position with Ar¹ in General Formula (1).

L^{1a} and L^{1b} each independently represent an alkylene group having 1 to 25 carbon atoms.

In General Formula (1), the aromatic hydrocarbon ring constituting the arylene group represented by Ar¹ may be any of a monocycle or a polycycle (a structure in which a plurality of monocycles are fused). In addition, the aromatic hydrocarbon ring may be a structure in which a plurality of aromatic hydrocarbon rings are bonded through a single bond, such as a biphenyl structure. That is, the arylene group may be a biphenyl group.

The number of carbon atoms in the arylene group is preferably 6 to 20, and more preferably 6 to 12.

Among these, as the arylene group, a group represented by any of Formulae (2-1) to (2-3) is preferable.

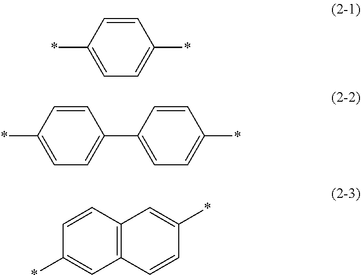

In addition, the specific compound is more preferably the compound represented by General Formula (2) or the compound represented by General Formula (3).

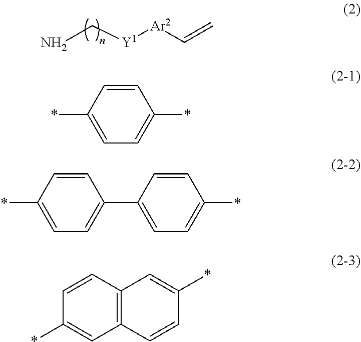

In General Formula (2),
Y¹ represents —O— or —OCH₂—.
Ar² represents a group represented by any of Formulae (2-1) to (2-3).
n represents an integer of 5 or more.
* represents a bonding position.

n is not particularly limited as long as it is 5 or more, but n is preferably 6 to 30, more preferably 8 to 28, and still more preferably 10 to 24.

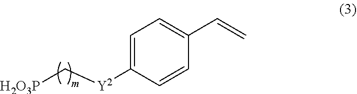

In General Formula (3),
Y² represents —O— or —OCH₂—.
m represents an integer of 2 or more.

m is not particularly limited as long as it is 2 or more, and is preferably 6 to 30, more preferably 8 to 28, and still more preferably 10 to 24.

In a case where the specific compound has two or more specific functional groups, examples of the specific compound include a compound represented by General Formula (S2).

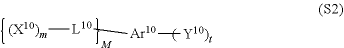

In General Formula (S2), X¹⁰ represents an amino group, a hydrazine group, a guanidine group, a phosphonic acid group, a sulfo group, or a carboxy group.

Specific aspects and suitable aspects of each group represented by X¹⁰ are as described above for the specific functional group. Among these, a primary amino group, the phosphonic acid group, or the sulfo group is preferable as the group represented by X¹⁰.

m represents an integer of 1 to 3, and is preferably 1 or 2, and more preferably 1.

In General Formula (S2), L¹⁰ represents an (m+1)-valent linking group or a single bond. As L¹⁰, the (m+1)-valent linking group is preferable. A preferred range of m is as described above.

Examples of the (m+1)-valent linking group represented by L¹⁰ include an aliphatic hydrocarbon group which may have at least one linking group selected from the group consisting of an etheric oxygen atom (—O—), a nitrogen atom (—N═), —NRN-(where R^{N} represents a hydrogen atom or an alkyl group having 1 to 6 carbon atoms), and a carbonyl group (—CO—), an aromatic ring group, and a group formed by a combination thereof. Among these, the aliphatic hydrocarbon group which may have at least one linking group is preferable.

The aliphatic hydrocarbon group represented by L¹⁰ is preferably a divalent or trivalent aliphatic hydrocarbon group which may have at least one linking group.

Examples of the divalent aliphatic hydrocarbon group which may have at least one linking group include a divalent aliphatic hydrocarbon group —O—.

Examples of the trivalent aliphatic hydrocarbon group which may have at least one linking group include —O-divalent aliphatic hydrocarbon group —N(-divalent aliphatic hydrocarbon group)₂,—N (-divalent aliphatic hydrocarbon group)₂, and a trivalent aliphatic hydrocarbon group. The “divalent aliphatic hydrocarbon groups” may be the same as or different from each other.

The number of carbon atoms in the aliphatic hydrocarbon group is preferably 1 to 20, more preferably 2 to 18, and still more preferably 6 to 18.

In addition, as the aliphatic hydrocarbon group, an alkylene group is preferable.

The aromatic ring group represented by L¹⁰ is preferably a divalent or trivalent aromatic ring group. Examples of the divalent aromatic ring group include a phenylene group. In addition, examples of the trivalent aromatic ring group include a group obtained by removing three hydrogen atoms from a benzene ring.

In General Formula (S2), M represents an integer of 2 or more, and is preferably 2 to 4, more preferably 2 or 3, and still more preferably 2. Furthermore, it is noted that a plurality of X¹⁰'s, a plurality of L¹⁰'s, and a plurality of m's may be the same as or different from each other, but are often the same.

In General Formula (S2), Ar¹⁰ represents an (M+t)-valent aromatic ring group. As Ar¹⁰, a group obtained by removing (M+t) pieces of hydrogen atoms from benzene, biphenyl, or naphthalene is preferable, and a group represented by any of Formulae (5-1) to (5-3) is more preferable.

In General Formula (S2), Y¹⁰ represents an ethylenically unsaturated group.

Examples of the ethylenically unsaturated group represented by Y¹⁰ include those exemplified as the polymerizable group, and among these, a vinyl group, a styryl group, or an acrylamide group is preferable.

In General Formula (S2), t represents 1 or 2, and is preferably 1.

A plurality of Y¹⁰'s may be the same as or different from each other, but are often the same.

In a case where the specific compound has two or more specific functional groups, the specific compound is preferably a compound represented by General Formula (4), and more preferably a compound represented by General Formula (5) which will be described later.

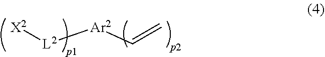

In General Formula (4),
X² represents a primary amino group, a phosphonic acid group, or a sulfo group.
L² represents an alkylene group which may have an etheric oxygen atom. The alkylene group is preferably a linear alkylene group. The number of carbon atoms in the alkylene group which may have an etheric oxygen atom, represented by L², is preferably 1 to 20, more preferably 2 to 18, and still more preferably 6 to 18.
p1 represents an integer of 2 or more, and is preferably 2 to 4, more preferably 2 or 3, and still more preferably 2.
p2 represents 1 or 2, and is preferably 1.
Ar² represents a (p1+p2)-valent aromatic ring group.
As the (p1+p2)-valent aromatic ring group represented by Ar², a group obtained by removing (p1+p2) pieces of hydrogen atoms from an aromatic hydrocarbon is preferable. As the aromatic hydrocarbon, benzene, biphenyl, or naphthalene is preferable.

In a case of p1=2 and p2=1, Ar² is more preferably a group represented by any of Formulae (5-1) to (5-3).

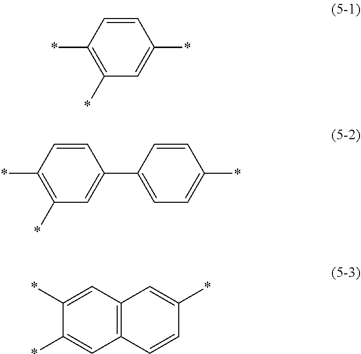

Hereinafter, General Formula (5) will be described in detail.

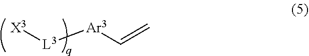

In General Formula (5),
X³ represents a primary amino group or a phosphonic acid group.
L³ represents —(CH₂)ᵣO— or an alkylene group having 6 to 18 carbon atoms.
r represents an integer of 2 to 18. r may be 2 to 18, but is preferably 6 to 18, and more preferably 8 to 12.
Ar³ represents a group obtained by removing (q+1) pieces of hydrogen atoms from an aromatic hydrocarbon selected from the group consisting of benzene, biphenyl, and naphthalene. q represents an integer of 2 or more, and is preferably 2 to 4, more preferably 2 or 3, and still more preferably 2.

Among these, Ar³ is preferably a group represented by any of Formulae (5-1) to (5-3).

The molecular weight of the specific compound is not particularly limited, but is preferably 200 to 1,000, more preferably 250 to 900, still more preferably 300 to 800, and particularly preferably 350 to 700.

The content of the specific compound is preferably 10.00% by mass or less, more preferably 3.00% by mass or less, and still more preferably 1.00% by mass or less with respect to the total mass of the present composition.

The lower limit is preferably 0.0001% by mass or more, more preferably 0.001% by mass or more, still more preferably 0.01% by mass or more, and particularly preferably 0.05% by mass or more.

Two or more kinds of the specific compounds may be used in combination.

In a case where two or more specific compounds are used in combination, a total content thereof is preferably within the range.

### (Solvent)

The present composition includes a solvent.

Examples of the solvent include water and an organic solvent.

Examples of the organic solvent include a hydrocarbon-based solvent, an alcohol-based solvent, a polyol-based solvent, a glycol ether-based solvent, an ether-based solvent, a ketone-based solvent, an amide-based solvent, a sulfur-containing solvent, and an ester-based solvent.

Examples of the hydrocarbon-based solvent include an aliphatic hydrocarbon-based solvent such as n-pentane and n-hexane; an alicyclic hydrocarbon-based solvent such as cyclohexane and methylcyclohexane; and an aromatic hydrocarbon-based solvent such as toluene and xylene.

Examples of the alcohol-based solvent include an aliphatic alcohol-based solvent having 1 to 18 carbon atoms such as methanol, ethanol, 1-propanol, 2-propanol (also referred to as isopropyl alcohol (IPA)), 2-butanol, isobutyl alcohol, tert-butyl alcohol, isopentyl alcohol, and 4-methyl-2-pentanol (also referred to as methyl isobutyl carbinol (MIBC)); an alicyclic alcohol-based solvent having 3 to 18 carbon atoms such as cyclohexanol; an aromatic alcohol-based solvent such as benzyl alcohol; and a ketone alcohol-based solvent such as diacetone alcohol.

The number of carbon atoms in the alcohol-based solvent is preferably 1 to 8, more preferably 2 to 7, and still more preferably 3 to 6.

Examples of the polyol-based solvent include a glycol-based solvent having 2 to 18 carbon atoms.

Examples of the glycol-based solvent include ethylene glycol, propylene glycol (1,2-propanediol), 1,3-propanediol, diethylene glycol, and dipropylene glycol.

Examples of the glycol ether-based solvent include a glycol monoether-based solvent having 3 to 19 carbon atoms.

Examples of the glycol monoether-based solvent include ethylene glycol monomethyl ether, ethylene glycol monoethyl ether, ethylene glycol mono-n-propyl ether, ethylene glycol monoisopropyl ether, ethylene glycol mono-n-butyl ether, diethylene glycol monomethyl ether, diethylene glycol monoethyl ether, diethylene glycol monobutyl ether, triethylene glycol monomethyl ether, triethylene glycol monoethyl ether, triethylene glycol monobutyl ether, 1-methoxy-2-propanol, 2-methoxy-1-propanol, 1-ethoxy-2-propanol, 2-ethoxy-1-propanol, propylene glycol monomethyl ether, propylene glycol mono-n-propyl ether, dipropylene glycol monomethyl ether, dipropylene glycol monoethyl ether, dipropylene glycol mono-n-propyl ether, tripropylene glycol monoethyl ether, tripropylene glycol monomethyl ether, ethylene glycol monobenzyl ether, and diethylene glycol monobenzyl ether.

The number of carbon atoms in the glycol ether-based solvent is preferably 1 to 8, more preferably 2 to 7, and still more preferably 3 to 6.

Examples of the ketone-based solvent include acetone, methyl ethyl ketone, methyl isobutyl ketone, and cyclohexanone.

Examples of the ether-based solvent include diethyl ether, diisopropyl ether, dibutyl ether, t-butyl methyl ether, cyclohexyl methyl ether, and tetrahydrofuran.

Examples of the amide-based solvent include formamide, monomethylformamide, dimethylformamide, acetamide, monomethylacetamide, dimethylacetamide, monoethylacetamide, diethylacetamide, and N-methylpyrrolidone.

Examples of the sulfur-containing solvent include dimethyl sulfone, dimethyl sulfoxide, and sulfolane.

Examples of the ester-based solvent include n-butyl acetate, ethyl lactate, propylene glycol acetate, propylene glycol monomethyl ether acetate, γ-butyrolactone, and δ-valerolactone.

Among these, as the solvent, the organic solvent is preferable, and the glycol monoether-based solvent or the alcohol-based solvent is more preferable.

The content of the solvent in the present composition is preferably 90.00% to 99.9999% by mass, more preferably 97.00% to 99.999% by mass, and still more preferably 99.00% to 99.99% by mass with respect to the total mass of the composition.

In addition, the total amount of the specific compound and the solvent is preferably 95.00% by mass or more, more preferably 99.00% by mass or more, and still more preferably 99.90% by mass or more with respect to the total mass of the present composition. The upper limit is not particularly limited, and may be, for example, 100% by mass or less.

In a case where the total amount of the specific compound and the solvent is within the range, the amount of components other than the specific compound and the solvent is extremely small, and therefore, it is possible to efficiently form a film having less impurities in the present composition and consisting of the specific compound on the substrate.

Two or more kinds of the solvents may be used in combination.

In a case where two or more kinds of the solvents are used in combination, a total content thereof is preferably within the range.

### (Polymerization Inhibitor)

From the viewpoint of improving the stability of the present composition, it is preferable that the present composition includes a polymerization inhibitor.

The polymerization inhibitor is not particularly limited, and a known polymerization inhibitor may be selected according to the kind of the crosslinkable group (polymerizable group) contained in the specific compound. However, a radical polymerization inhibitor is preferable.

The polymerization inhibitor preferably includes at least one compound selected from the group consisting of a phenol-based compound, a quinone-based compound, a free radical-based compound, an amine-based compound, and a phosphine-based compound, and from the viewpoint of polymerization inhibition ability, the free radical-based compound is more preferable.

Examples of the phenol-based compound include 4-methoxyphenol, hydroquinone, 2-tert-butylhydroquinone, 4-tert-butylcatechol, pentaerythritol tetrakis[3-(3,5-di-tert-butyl-4-hydroxyphenyl)propionate], 2,5-di-tert-butyl-4-methylphenol, 2,6-di-tert-butyl-4-methylphenol, 4,4′-thiobis(3-methyl-6-t-butylphenol), 2,2′-methylenebis(4-methyl-6-t-butylphenol), 4-methoxynaphthol, 2,4-bis(octylthiomethyl)-6-methylphenol, p-nitrosophenol, and α-nitroso-β-naphthol.

Examples of the quinone-based compound include 1,4-benzoquinone, 1,2-benzoquinone, and 1,4-naphthoquinone.

Examples of the free radical-based compound include poly (4-methacryloyloxy-2,2,6,6-tetramethylpiperidine-N-oxyl), 4-hydroxy-2,2,6,6-tetramethylpiperidine 1-oxyl, 2,2,6,6-tetramethylpiperidine 1-oxyl, 2,2-diphenyl-1-picrylhydrazyl, and triphenylverdazyl.

Examples of the amine-based compound include p-phenylenediamine, 4-aminodiphenylamine, N,N-diethylhydroxylamine, N,N′-diphenyl-p-phenylenediamine, N-isopropyl-N′-phenyl-p-phenylenediamine, N-(1,3-dimethylbutyl)-N′-phenyl-p-phenylenediamine, N,N′-di-2-naphthyl-p-phenylenediamine, diphenylamine, N-phenyl-β-naphthylamine, 4,4′-dicumyl-diphenylamine, 4,4′-dioctyl-diphenylamine, phenothiazine, 2-methoxyphenothiazine, phenoxazine, N-nitrosodiphenylamine, N-nitrosophenylnaphthylamine, N-nitrosodinaphthylamine, p-nitrosodiphenylamine, N-nitroso-N-phenylhydroxylamine, N-nitroso-N-phenylhydroxylamine aluminum, and cupferron. Each of the compounds exemplified as the amine-based compound may form a metal salt or a metal complex.

Examples of the phosphine-based compound include tris(2,4-di-tert-butylphenyl) phosphite.

In addition, as the polymerization inhibitor, nitrobenzene-based compounds such as nitrobenzene and 4-nitrotoluene, and thiol ethers such as dioctadecyl 3,3′-thiodipropionate, dilauryl thiodipropionate, dimyristyl thiodipropionate, and distearyl thiodipropionate may also be included.

The molecular weight of the polymerization inhibitor is preferably 1,000 or less, more preferably 800 or less, and still more preferably 500 or less. The lower limit of the molecular weight is not particularly limited, but is preferably 80 or more.

The content of the polymerization inhibitor is preferably 0.0001 parts by mass or more, more preferably 0.001 parts by mass or more, still more preferably 0.005 parts by mass or more, and particularly preferably 0.010 parts by mass or more with respect to 100 parts by mass of the content of the specific compound.

In addition, the content of the polymerization inhibitor is preferably 10.0 parts by mass or less, more preferably 1.000 part by mass or less, and still more preferably 0.100 parts by mass or less with respect to 100 parts by mass of the content of the specific compound.

The present composition may include one kind of polymerization inhibitor alone, or may include two or more kinds of the polymerization inhibitors. In a case where the composition includes two or more kinds of the polymerization inhibitors, a total amount thereof is preferably within the range.

### Method for Producing Composition

A method for producing the composition for treating a semiconductor device of the embodiment of the present invention is not particularly limited, and the composition can be produced, for example, by mixing the above-described components.

The order or timing of mixing the respective components in the composition is not particularly limited. Examples of the method include a method for producing a composition by adding the specific compound to a stirrer such as a mixing mixer into which a purified solvent has been incorporated, and then sufficiently stirring the mixture.

In a case where the composition includes other components in addition to the specific compound, the other components may be added at the same time as the specific compound or may be added at different timings.

In the production step of producing the composition, the steps described below may be carried out.

### (Metal Removing Step)

In the production method, a metal removal step of removing a metal component from the components and/or composition (hereinafter also referred to as a “substance to be purified”) may be performed.

### (Filtration Step)

It is preferable that the production method include a filtration step of filtering a liquid such that foreign substances, coarse particles, and the like are removed from the liquid.

The filtration method is not particularly limited, and a publicly known filtration method can be used. Among these, filtering using a filter is preferable.

### (Static Neutralization Step)

The method for producing a composition may further include a static neutralization step of statically neutralizing the composition.

### Use

The present composition is a composition for treating a semiconductor device. In the present specification, the expression, “for treating a semiconductor device”, means that it is used in the production of a semiconductor device. The present composition can be used in any step for producing a semiconductor device, and for example, it can be used in a step of treating a semiconductor substrate, which is included in a method for producing a semiconductor device.

More specifically, the present composition is preferably used in a method for producing a modified substrate, which will be described in detail later.

### Method for Treating Substrate

As described above, the present composition can be used in a step of treating a semiconductor substrate, and the present composition can typically be used by bringing the present composition into contact with an object to be treated (in particular, a substrate having a surface including metal atoms) including metal atoms. In this case, the object to be treated may include a plurality of kinds of metal atoms.

The contact angle of a film obtained by applying the present composition with respect to water is preferably 60 degrees or more, more preferably 90 degrees or more, and still more preferably 105 degrees or more. The upper limit is not particularly limited, and is often 120 degrees or less.

The contact angle of the film obtained by applying the present composition with respect to water can be measured using, for example, a fully automatic contact angle meter DMo-901 (manufactured by Kyowa Interface Science Co., Ltd.).

### (Substrate)

A substrate in a method for treating a substrate using the present composition (hereinafter also simply referred to as “the present treatment method”) is not particularly limited, but it is preferable that the substrate has at least two surfaces of a first surface and a second surface, which are composed of materials different from each other. In a case of having the two surfaces, it is possible to form a coating film having high inhibitory properties for an ALD coating film on one surface by selectively bonding or adsorbing a specific compound onto one surface.

It is preferable that a substrate having at least two surfaces of a first surface and a second surface is treated with the present composition to form a coating film having high inhibitory properties for an ALD coating film on the first surface and to selectively form a coating film formed by an ALD treatment on the second surface.

The material constituting the first surface and the material constituting the second surface are not particularly limited as long as they are different from each other, and may be any of an organic material or an inorganic material. However, from the viewpoint that the effect of the present invention is more excellent, it is preferable that at least one of the first surface or the second surface includes a metal atom, and it is more preferable that the first surface includes a metal atom.

Furthermore, in the present specification, metalloid atoms such as boron, silicon, germanium, arsenic, antimony, and tellurium are also included in the metal atoms.

In a case where the first surface or the second surface includes a metal atom, the metal atom may be included, for example, as a metal (for example, a simple metal) or a metal atom included in a compound. In addition, the metal atom may be included as a metal atom that is included in a pure metal or an alloy.

As the metal atom, a transition metal atom is preferable, at least one metal atom selected from the group consisting of a copper atom, a cobalt atom, a titanium atom, a tantalum atom, a tungsten atom, a ruthenium atom, and a molybdenum atom is preferable, at least one metal atom selected from the group consisting of the titanium atom, the tungsten atom, the ruthenium atom, and the molybdenum atom is more preferable, and the ruthenium atom or the tungsten atom is still more preferable.

In addition, examples of other preferred aspects of the first surface and the second surface include an aspect in which one of the first surface and the second surface is a metal surface composed of a metal and the other is a non-metal surface composed of a non-metal (hereinafter also referred to as an aspect A).

In the aspect A, it is preferable that the first surface is a metal surface and the second surface is a non-metal surface.

Examples of the metal include a pure metal or an alloy.

Examples of the non-metal include a metal carbide, a metal oxide, a metal nitride, a metal oxynitride, and an organic material.

It is preferable that the pure metal and the alloy are composed of the preferred metal atoms exemplified above.

In addition, it is preferable that the metal carbide, the metal oxide, the metal nitride, and the metal oxynitride are the metal carbide, the metal oxide, the metal nitride, and the metal oxynitride, which are preferable metal atoms as exemplified above.

In addition, examples of a preferred aspect of the first surface and the second surface include an aspect in which the first surface is a surface composed of a material selected from the group consisting of a metal (a pure metal or an alloy), a metal carbide, a metal oxide, a metal nitride, and a metal oxynitride, and the second surface is a surface composed of a material different from that of the first surface and selected from the group consisting of a metal (a pure metal or an alloy), a metal carbide, a metal oxide, a metal nitride, and a metal oxynitride (hereinafter also referred to as an aspect B).

The expression that the first surface and the second surface are composed of different kinds of materials is intended to mean that two kinds of materials are selected as the first surface and the second surface from five kinds of materials of a metal, a metal carbide, a metal oxide, a metal nitride, and a metal oxynitride.

More specific examples of the aspect B include an aspect in which the first surface is a metal surface composed of a metal and the second surface is a metal oxide surface composed of a metal oxide (hereinafter also referred to as an aspect B1), an aspect in which the first surface is a metal nitride surface composed of a metal nitride and the second surface is a metal oxide surface composed of a metal oxide (hereinafter also referred to as an aspect B2), and an aspect in which the first surface is a metal oxide surface composed of a metal oxide and the second surface is a metal surface composed of a metal (hereinafter also referred to as an aspect B3).

In a case of the aspect B1, examples of the metal include copper, cobalt, titanium, tantalum, tungsten, ruthenium, and molybdenum.

Examples of the metal oxide include silicon oxide and tetraethyl orthosilicate (TEOS).

In a case of the aspect B2, examples of the metal nitride include titanium nitride.

Examples of the metal oxide include silicon oxide and tetraethyl orthosilicate (TEOS).

In a case of the aspect B3, examples of the metal oxide include silicon oxide and tetraethyl orthosilicate (TEOS).

Examples of the metal include silicon.

### Method for Producing Modified Substrate

As one of the methods for treating a substrate, a method for producing a modified substrate, using the present composition and a substrate having at least two surfaces of a first surface and a second surface, which are composed of materials different from each other, is suitably mentioned.

The method for producing a modified substrate preferably includes a step 1 to a step 3, and may include a step 4 as necessary.
Step 1: A step of bringing a substrate having at least two surfaces of a first surface and a second surface, which are composed of materials different from each other, into contact with the present composition to form a first film on the first surface
Step 2: A step of subjecting the substrate obtained in the step 1 to an atomic layer deposition treatment (ALD treatment) to form a second film on the second surface
Step 3: A step of removing the first film formed on the substrate after the step 2
Step 4: A step of heating the modified substrate obtained in the step 3

### Step 1

The step 1 is a step of bringing a substrate having at least two surfaces of a first surface and a second surface, which are composed of materials different from each other (hereinafter also simply referred to as a “specific substrate”), into contact with the present composition to form a first film on the first surface. The first film is a film including a specific compound.

A method for bringing the specific substrate into contact with the present composition is not particularly limited, and examples thereof include a method of applying or spraying the present composition onto the specific substrate and a method of immersing the specific substrate in the present composition. A method for applying the present composition onto the specific substrate is not particularly limited, a known method can be used, and examples of the method include a spin coating method. In addition, in a case where the specific substrate is immersed in the present composition, the present composition may be allowed to flow.

The temperature of the present composition in a case of bringing the specific substrate into contact with the present composition is not particularly limited, but is preferably 0° C. to 50° C., and more preferably 10° C. to 30° C.

It is also preferable that the specific substrate and the present composition are brought into contact with each other, and the specific substrate on which the first film is formed on the first surface is subjected to a rinsing treatment. The specific compound adhering to a region other than a desired region on the specific substrate can be removed from the substrate by the rinsing treatment.

The rinsing method is not particularly limited, and examples thereof include a method of bringing a rinsing liquid into contact with the specific substrate. Examples of the contact method include the same method as the method for bringing the present composition into contact with the specific substrate. The temperature of the rinsing liquid during the contact is not particularly limited, but is preferably 0° C. to 50° C., and more preferably 10° C. to 30° C.

The rinsing liquid is not particularly limited, and examples thereof include the solvent included in the present composition. A solvent of the same type as the solvent included in the present composition may be used as the rinsing liquid.

### Step 2

The step 2 is a step of subjecting the specific substrate obtained in the step 1 (the specific substrate having the first film) to an atomic layer deposition treatment (ALD treatment) to form a second film on the second surface.

In a case where the specific substrate obtained in the step 1 is subjected to the ALD treatment, the reaction of the polymerizable group of the specific compound in the first film proceeds to cure the film, and the formation of the second film is inhibited by the cured first film. Therefore, a modified substrate in which the second film (ALD coating film) is formed with good selectivity on a region (second surface) where the first film is not formed can be obtained.

Since the material used for forming a Langmuir-Blodgett film or a self-assembled monolayer film (SAM film) is often a low-molecular-weight material, the material is very effective for selectively forming a modified film on a fine region on a substrate, as compared with a high-molecular-weight material, but the material is often inferior in resistance in a case where the substrate is heated in the ALD treatment.

In contrast, in the step 2, since the specific compound is a low-molecular-weight material but has a possible group, in a case where the substrate is heated in the ALD step as described above, the first film is cured to form a cured film, and sufficient heat resistance can be exhibited, making it possible to inhibit the formation of an ALD coating film in a fine region while maintaining the heat resistance.

In the ALD treatment, a precursor serving as a raw material of the second film is supplied to a surface of the specific substrate obtained in the step 1. It is general to use two or more kinds of the precursors.

The material constituting the second film can be controlled by the kind of a precursor to be supplied, the supply atmosphere, an oxidant, and the like. The second film formed by the ALD treatment is not particularly limited, but is preferably a metal film, a metal oxide film, or a metal nitride film, and more preferably the metal film or the metal oxide film.

Examples of the metal constituting the metal film include aluminum, titanium, chromium, iron, cobalt, nickel, copper, zinc, yttrium, zirconium, niobium, molybdenum, ruthenium, palladium, lanthanum, cerium, hafnium, tantalum, tungsten, platinum, and bismuth.

Examples of the metal oxide constituting the metal oxide film include aluminum oxide, titanium oxide, zinc oxide, zirconium oxide, hafnium oxide, and tantalum oxide.

Examples of the metal nitride constituting the metal nitride film include titanium nitride and tantalum nitride.

In addition, in the ALD treatment, a treatment for altering a surface of the region where the first film is not formed may be performed.

The ALD treatment is not particularly limited, but a thermal ALD method is preferable.

From the viewpoint that the reaction between the polymerizable groups can be efficiently performed, the substrate heating temperature in the ALD treatment is preferably 100° C. to 400° C., more preferably 150° C. to 400° C., and still more preferably 200° C. to 300° C.

In the step 2, a difference between the thickness of the second film on the second surface and the thickness of the second film on the region where the first film is formed (the thickness of the second film on the second surface-the thickness of the second film on the region where the first film is formed) is preferably 1.0 nm or more, more preferably 1.2 nm or more, and still more preferably 1.5 nm or more.

The upper limit of the difference in thickness is not particularly limited, but may be, for example, 100 nm or less.

### Step 3

The method for producing a modified substrate may include a step 3 of removing the first film formed on the specific substrate in the step 1 after the step 2. By carrying out the step 3 after the step 2, a modified substrate in which the second film is formed only on the second surface is obtained.

A method for removing the first film is not particularly limited, and examples thereof include dry etching, wet etching, and a combination thereof.

Examples of the dry etching include a method of supplying reactive ions or reactive radicals to the surface of the modified substrate having the first film. The reactive ions or the reactive radicals may be generated by plasma or the like, and are preferably generated using a mixed gas including one or more gases selected from the group consisting of oxygen, nitrogen, and hydrogen. The mixed gas may include a rare gas. In addition, the dry etching may be physical etching using a sputtering phenomenon.

In the wet etching, an etchant may be supplied to a surface of the modified substrate having the first film. Examples of the etchant include an etchant including an oxidant such as ozone and an etchant including an organic solvent. Examples of the organic solvent in the etchant including an organic solvent include the organic solvent contained in the composition, and the hydrocarbon-based solvent is preferable.

### Step 4

The method for producing a modified substrate may include a step 4 of heating the modified substrate. It is preferable that the step 4 is carried out before the step 2. By carrying out the step 4, the polymerizable groups can react with each other.

The heating temperature is not particularly limited, but is preferably 100° C. to 400° C., more preferably 150° C. to 400° C., and still more preferably 200° C. to 300° C.

The heating method is not particularly limited, and examples thereof include a method of contacting with a heating element (for example, heating with a hot plate) and a method of irradiation with infrared rays.

### Compound

The present invention also includes a compound. The compound of the embodiment of the present invention is the compound represented by General Formula (2), the compound represented by General Formula (3), or the compound represented by General Formula (5).

### EXAMPLES

Hereinbelow, the present invention will be described in more detail with reference to Examples.

The materials, the amounts of materials used, the proportions, the treatment details, the treatment procedure, and the like shown in Examples below may be modified as appropriate as long as the modifications do not depart from the spirit of the present invention. Therefore, the scope of the present invention should not be construed as being limited to Examples shown below.

Among the specific compounds E-1 to E-22 used in each Example, E-1, E-4, E-6, E-8, E-9, E-10, E-11, E-12, E-14, and E-21 were synthesized with reference to any of the following Synthesis Examples 1 to 5.

In addition, among the specific compounds E-23 to E-35 used in each Example, E-25, E-26, E-30, E-31, and E-32 were synthesized with reference to any of the following Synthesis Examples 6 and 7.

For the specific compounds other than those above, a commercially available product or a synthetic product obtained according to a known procedure was used.

### Synthesis of Specific Compound E-9 (Synthesis Example 1)

A specific compound E-9 was synthesized according to the following scheme.

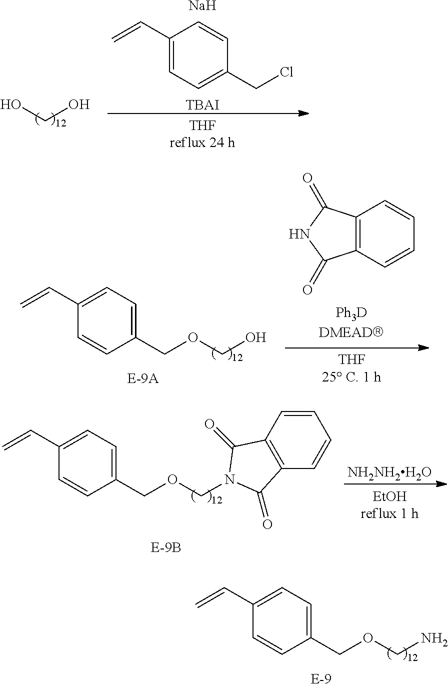

### Synthesis of Intermediate E-9A

Under a nitrogen flow (0.1 L/min), 1,12-dodecanediol (50.0 g, 0.25 mol, manufactured by Tokyo Chemical Industry Co., Ltd.) and tetrahydrofuran (THF, 1.0 L, manufactured by FUJIFILM Wako Pure Chemical Corporation) were added to a three-neck flask, and the mixture was cooled to 0° C. Then, 60% sodium hydride (10.9 g, 0.27 mol, manufactured by Kanto Chemical Co., Inc.) was gradually added and the mixture was further stirred at 0° C. for 1 hour. 4-(Chloromethyl)styrene (41.5 g, 0.27 mol, manufactured by Tokyo Chemical Industry Co., Ltd.) and tetrabutylammonium iodide (TBAI, 16.7 g, 0.03 mol, manufactured by Tokyo Chemical Industry Co., Ltd.) were added to the mixed solution after the completion of the stirring, and the obtained reaction solution was heated and refluxed for 24 hours with stirring.

The reaction solution was cooled to 0° C., water was added thereto to quench the reaction, and the insoluble matter was removed by filtration. The solvent was distilled off from the filtrate under a reduced pressure of 40° C./10 hPa, ethyl acetate (500 mL, manufactured by FUJIFILM Wako Pure Chemical Corporation) and distilled water (500 mL) were added thereto, and the obtained solution was transferred to a separating funnel and stirred. Thereafter, the solution was allowed to stand to remove the lower phase (water phase) and the upper phase (organic phase) was recovered. The solvent was distilled off from the obtained organic phase under a reduced pressure of 40° C./10 hPa.

The obtained crude product was purified by silica gel column chromatography to obtain an intermediate E-9A.

### Synthesis of Intermediate E-9B

Under a nitrogen flow (50 mL/min), the intermediate E-9A (17.6 g, 0.06 mol), triphenylphosphine (21.7 g, 0.08 mol, manufactured by FUJIFILM Wako Pure Chemical Corporation), phthalimide (12.2 g, 0.08 mol, manufactured by Tokyo Chemical Industry Co., Ltd.), and THF (220 mL, manufactured by FUJIFILM Wako Pure Chemical Corporation) were added to a three-neck flask, and the obtained reaction solution was cooled to 0° C.

Next, a dissolving solution obtained by dissolving bis(2-methoxyethyl) azodicarboxylate (DMEAD (registered trademark), 19.4 g, 0.08 mol, manufactured by FUJIFILM Wako Pure Chemical Corporation) in THF (110 mL) was separately prepared.

While maintaining the internal temperature of the reaction solution obtained above at 5° C. or lower, the dissolving solution was added dropwise to the reaction solution over 2 hours. After completion of the dropwise addition, the reaction solution was stirred at 25° C. for 1 hour.

Next, the solvent was distilled off from the reaction solution under a reduced pressure of 40° C./10 hPa. After the distillation, ethyl acetate (500 mL, manufactured by FUJIFILM Wako Pure Chemical Corporation) and a 1 M aqueous sodium hydroxide solution (250 mL) were added thereto, and the obtained solution was transferred to a separating funnel and stirred. Thereafter, the solution was allowed to stand to remove the lower phase (water phase), and distilled water (400 mL) was added to the upper phase (organic phase) and stirred. Further, the solution was then allowed to stand to remove the lower phase (water phase), and the upper phase (organic phase) was recovered. The solvent was distilled off from the obtained organic phase under a reduced pressure of 40° C./10 hPa.

The obtained crude product was purified by silica gel column chromatography to obtain an intermediate E-9B.

### Synthesis of Specific Compound E-9

Under a nitrogen flow (0.1 L/min), the intermediate E-9B (20.3 g, 0.05 mol) and ethanol (406 mL, manufactured by FUJIFILM Wako Pure Chemical Corporation) were added to a three-neck flask, and stirred. Hydrazine monohydrate (9.1 g, 0.18 mmol, manufactured by FUJIFILM Wako Pure Chemical Corporation) was further added to the obtained mixed solution and the mixture was heated under reflux for 1 hour. Thereafter, the obtained reaction solution was cooled to 0° C., and the precipitate was removed by filtration. The solvent was distilled off from the filtrate under a reduced pressure at 40° C./10 hPa, tert-butyl methyl ether (260 mL, manufactured by FUJIFILM Wako Pure Chemical Corporation) and a 1 M aqueous sodium hydroxide solution (130 mL) were added to the obtained crude product, and the obtained solution was transferred to a separating funnel and stirred. Thereafter, the solution was allowed to stand to remove the lower phase (water phase) and the upper phase (organic phase) was recovered. The obtained organic phase was concentrated to obtain a specific compound E-9.

The ¹H-nuclear magnetic resonance (NMR) data of the obtained specific compound E-9 are shown below.

¹H-NMR (400 MHZ, DMSO-d6): δ(ppm)=7.44 (d, J=8.1 Hz, 2H), 7.28 (d, J=8.1 Hz, 2H), 6.72 (dd, J=10.9 Hz, 17.6 Hz, 1H), 5.81 (d, J=17.6 Hz, 1H), 5.24 (d, J=10.9 Hz, 1H), 4.42 (s, 2H), 3.40 (t, J=6.5 Hz, 2H), 2.46-2.53 (m, 2H), 1.47-1.57 (m, 2H), 1.12-1.42 (m, 18H)

### Synthesis of Specific Compound E-10 (Synthesis Example 2)

The specific compound E-10 was synthesized in the same manner as in E-9, except that 1,16-hexadecanediol (50.0 g, 0.25 mol, manufactured by Tokyo Chemical Industry Co., Ltd.) was used instead of 1,12-dodecanediol.

The ¹H-NMR data of the obtained specific compound E-10 are shown below.

¹H-NMR (400 MHZ, DMSO-d6): δ(ppm)=7.44 (d, J=8.0 Hz, 2H), 7.28 (d, J=8.0 Hz, 2H), 6.72 (dd, J=11.0 Hz, 17.6 Hz, 1H), 5.81 (d, J=17.6 Hz, 1H), 5.24 (d, J=11.0 Hz, 1H), 4.42 (s, 2H), 3.40 (t, J=6.5 Hz, 2H), 2.46-2.53 (m, 2H), 1.47-1.57 (m, 2H), 1.12-1.42 (m, 26H)

### Synthesis of Specific Compound E-8 (Synthesis Example 3)

A specific compound E-8 was synthesized in the same manner as in E-9, except that 1,5-pentanediol (50.0 g, 0.25 mol, manufactured by Tokyo Chemical Industry Co., Ltd.) was used instead of 1,12-dodecanediol.

The ¹H-NMR data of the obtained specific compound E-8 are shown below.

¹H-NMR (400 MHZ, DMSO-d6): δ(ppm) =7.44 (d, J=8.0 Hz, 2H), 7.28 (d, J=8.0 Hz, 2H), 6.72 (dd, J=11.0 Hz, 17.6 Hz, 1H), 5.81 (d, J=17.6 Hz, 1H), 5.24 (d, J=11.0 Hz, 1H), 4.43 (s, 2H), 3.40 (t, J=6.5 Hz, 2H), 2.46-2.53 (m, 2H), 1.47-1.57 (m, 2H), 1.12-1.42 (m, 4H)

### Synthesis of Specific Compound E-1 (Synthesis Example 4)

A specific compound E-1 was synthesized according to the following scheme.

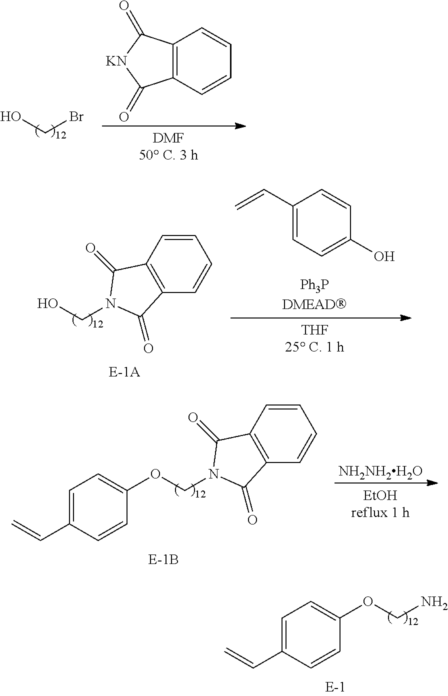

### Synthesis of Intermediate E-1A

Under a nitrogen flow (0.1 L/min), 12-bromo-1-dodecanol (50.0 g, 0.19 mol, manufactured by Tokyo Chemical Industry Co., Ltd.), dimethylformamide (DMF, 250 mL, manufactured by FUJIFILM Wako Pure Chemical Corporation), and phthalimide potassium (38.4 g, 0.21 mol, manufactured by Tokyo Chemical Industry Co., Ltd.) were added to a three-neck flask, and the obtained mixed solution was stirred at 50° C. for 3 hours.

After the obtained reaction solution was cooled to 25° C., the insoluble matter was removed by filtration and the filtrate was washed with DMF (50 mL) to obtain a filtrate A.

Distilled water (900 mL) was added to the beaker, the mixture was stirred at 25° C., and the filtrate A was added dropwise thereto. The obtained crystals were collected by filtration, washed twice with distilled water (200 mL), and then blast-dried at 40° C. for 24 hours to obtain an intermediate E-1A.

### Synthesis of Intermediate E-1B

Under a nitrogen flow (50 mL/min), the intermediate E-1A (50.0 g, 0.15 mol), triphenylphosphine (59.0 g, 0.23 mol, manufactured by FUJIFILM Wako Pure Chemical Corporation), 4-vinylphenol (27.0 g, 0.23 mol, manufactured by FUJIFILM Wako Pure Chemical Corporation), and tetrahydrofuran (THF, 500 mL, manufactured by FUJIFILM Wako Pure Chemical Corporation) were added to a three-neck flask, and the obtained reaction solution was cooled to 0° C.

Next, a dissolving solution obtained by dissolving bis (2-methoxyethyl) azodicarboxylate (DMEAD (registered trademark), 52.7 g, 0.23 mol, manufactured by FUJIFILM Wako Pure Chemical Corporation) in THF (158 mL) was separately prepared.

While maintaining the internal temperature of the reaction solution obtained above at 5° C. or lower, the dissolving solution was added dropwise to the reaction solution over 2 hours. After completion of the dropwise addition, the reaction solution was stirred at 25° C. for 1 hour.

Next, the solvent was distilled off from the reaction solution under a reduced pressure of 40° C./10 hPa. After the distillation, ethyl acetate (500 mL, manufactured by FUJIFILM Wako Pure Chemical Corporation) and a 1 M aqueous sodium hydroxide solution (250 mL) were added thereto, and the obtained solution was transferred to a separating funnel and stirred. Thereafter, the solution was allowed to stand to remove the lower phase (water phase), and distilled water (400 mL) was added to the upper phase (organic phase) and stirred. Further, the solution was then allowed to stand to remove the lower phase (water phase), and the upper phase (organic phase) was recovered. The solvent was distilled off from the obtained organic phase under a reduced pressure of 40° C./10 hPa.

The obtained crude product was purified by silica gel column chromatography to obtain an intermediate E-1B.

### Synthesis of Specific Compound E-1

Under a nitrogen flow (0.1 L/min), the intermediate E-1B (50.0 g, 0.12 mol) and ethanol (250 mL, manufactured by FUJIFILM Wako Pure Chemical Corporation) were added to a three-neck flask, and the mixture was stirred. Hydrazine monohydrate (23.1 g, 0.46 mmol, manufactured by FUJIFILM Wako Pure Chemical Corporation) was further added to the obtained mixed solution and the mixture was heated under reflux for 1 hour. Thereafter, the obtained reaction solution was cooled to 0° C., and the precipitate was removed by filtration. The solvent was distilled off from the filtrate under a reduced pressure at 40° C./10 hPa, tert-butyl methyl ether (250 mL, manufactured by FUJIFILM Wako Pure Chemical Corporation) and a 1 M aqueous sodium hydroxide solution (150 mL) were added to the obtained crude product, and the obtained solution was transferred to a separating funnel and stirred. Thereafter, the solution was allowed to stand to remove the lower phase (water phase) and the upper phase (organic phase) was recovered. The obtained organic phase was concentrated to obtain a specific compound E-1.

The ¹H-NMR data of the obtained specific compound E-1 are shown below.

¹H-NMR (400 MHZ, THF-d8): δ(ppm)=7.31 (d, J=8.7 Hz, 2H), 6.83 (d, J=8. 7 Hz, 2H), 6.62 (dd, J=10.9 Hz, 17.6 Hz, 1H), 5.57 (d, J=1.0 Hz, 17.6 Hz, 1H), 5.03 (d, J=1.0 Hz, 17.6 Hz, 1H), 3.94 (t, J=6.5 Hz, 2H), 2.59 (t, J=6.5 Hz, 2H), 1.70-1.80 (m, 2H), 1.42-1.52 (m, 2H), 1.22-1.42 (m, 16H)

### Synthesis of Specific Compound E-21 (Synthesis Example 5)

A specific compound E-21 was synthesized according to the following scheme.

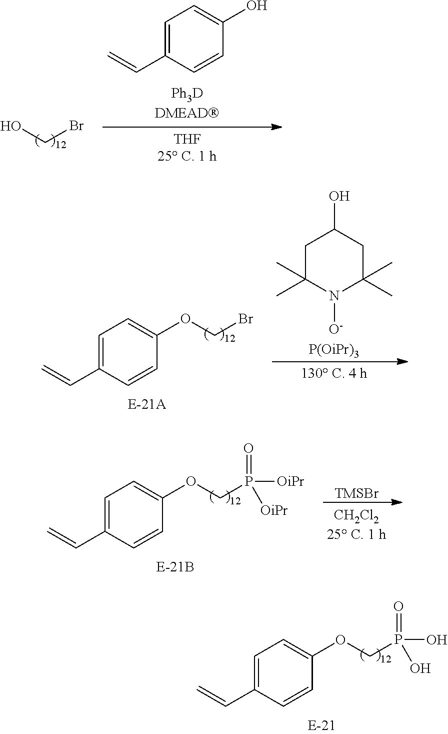

### Synthesis of Intermediate E-21A

Under a nitrogen flow (0.01 L/min), 12-bromo-1-dodecanol (15.0 g, 56.6 mmol, manufactured by Tokyo Chemical Industry Co., Ltd.), triphenylphosphine (22.3 g, 84.8 mol, manufactured by FUJIFILM Wako Pure Chemical Corporation), 4-vinylphenol (8.83 g, 73.5 mol), and THF (188 mL) were added to a three-neck flask, and the mixture was cooled to 0° C. Next, a dissolving solution obtained by dissolving bis (2-methoxyethyl) azodicarboxylate (DMEAD (registered trademark), 19.9 g, 84.8 mol, manufactured by FUJIFILM Wako Pure Chemical Corporation) in THF (94 mL) was separately prepared.

While maintaining the internal temperature of the reaction solution obtained above at 5° C. or lower, the above-described dissolving solution was added dropwise to the reaction solution over 4 hours. After completion of the dropwise addition, the reaction solution was stirred at 25° C. for 1 hour.

Next, the solvent was distilled off from the reaction solution under a reduced pressure of 40° C./10 hPa. After the distillation, ethyl acetate (500 mL, manufactured by FUJIFILM Wako Pure Chemical Corporation) and a 1 M aqueous sodium hydroxide solution (250 mL) were added thereto, and the obtained solution was transferred to a separating funnel and stirred. Thereafter, the solution was allowed to stand to remove the lower phase (water phase), and distilled water (400 mL) was added to the upper phase (organic phase) and stirred. Further, the solution was then allowed to stand to remove the lower phase (water phase), and the upper phase (organic phase) was recovered. The solvent was distilled off from the obtained organic phase under a reduced pressure of 40° C./10 hPa to obtain a 40% by mass ethyl acetate solution. Diisopropyl ether (260 mL, manufactured by FUJIFILM Wako Pure Chemical Corporation) was added to the obtained 40% by mass ethyl acetate solution, and the solution was cooled to 0° C. After stirring at 0° C. for 30 minutes, crystals were precipitated and the crystals were separated by filtration. The obtained crystals and methanol (412 mL, manufactured by Mitsubishi Gas Chemical Company, Inc.) were added to a glass eggplant flask and stirred at 25° C. for 1 hour. The obtained crystals were collected by filtration, washed with methanol, and then blast-dried at 40° C. for 12 hours to obtain an intermediate E-21A.

### Synthesis of Intermediate E-21B

The intermediate E-21A (10.0 g, 27.2 mmol), 4-hydroxy-TEMPO free radical (100 mg, 0.58 mmol, manufactured by Tokyo Chemical Industry Co., Ltd.), and triisopropyl phosphate (14.2 g, 68.2 mol, manufactured by Tokyo Chemical Industry Co., Ltd.) were added to a three-neck flask, and the obtained mixed solution was stirred at 130° C. for 4 hours.

Next, the excess triisopropyl phosphite used was distilled off from the reaction solution under a reduced pressure of 70° C./5 hPa and the obtained crude product was purified by silica gel column chromatography to obtain an intermediate E-21B.

### Synthesis of Specific Compound E-21

The intermediate E-21B (15.0 g, 33.1 mol) and dichloromethane (150 mL, manufactured by FUJIFILM Wako Pure Chemical Corporation) were added to a three-neck flask, and cooled to 0° C. Bromotrimethylsilane (TMSBr, 25.3 g, 165.2 mmol, manufactured by FUJIFILM Wako Pure Chemical Corporation) was added to the obtained mixed solution, the mixture was stirred at 0° C. for 4 hours, water (150 mL) was added to the reaction solution, and the mixture was stirred at 25° C. for 1 hour. The obtained solution was transferred to a separating funnel and the lower phase (organic phase) was recovered. The organic phase was concentrated and the obtained crude product was purified by silica gel column chromatography to obtain a specific compound E-21.

The ¹H-NMR data of the obtained specific compound E-21 are shown below.

¹H-NMR (400 MHZ, CDCl₃): δ(ppm)=7.31 (d, J=8.6 Hz, 2H), 6.83 (d, J=8.6 Hz, 2H), 6.64 (dd, J=10.9 Hz, 17.6 Hz, 1H), 5.57 (d, J=17.6 Hz, 1H), 5.09 (d, J=10.9 Hz, 1H), 3.90 (t, J=6.6 Hz, 2H), 1.12-1.72 (m, 22H)

### Synthesis of Specific Compound E-25 (Synthesis Example 6)

A specific compound E-25 was synthesized according to the following scheme.

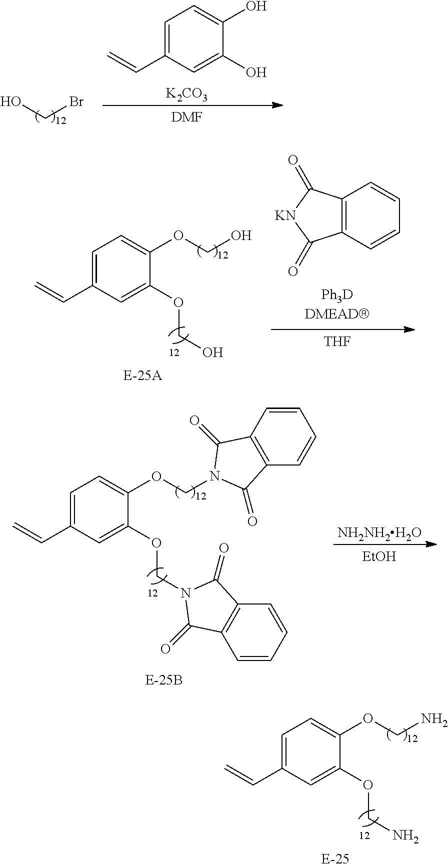

### Synthesis of Intermediate E-25A

Under a nitrogen flow (50 mL/min), 12-bromo-1-dodecanol (15.0 g, 56.6 mmol, manufactured by Tokyo Chemical Industry Co., Ltd.), potassium carbonate (23.4 g, 169.7 mmol, manufactured by Kanto Chemical Co., Inc.), 4-vinylcatechol (3.1 g, 22.6 mmol, manufactured by Henan Yufu New Materials Co., Ltd.), and DMF (150 mL) were added to a three-neck flask, and the mixture was heated to 90° C. After the temperature was raised, the reaction solution was stirred at 90° C. for 4 hours.

Next, the reaction solution was cooled to 25° C., the insoluble matter was removed by filtration, and the obtained filtrate was concentrated under a reduced pressure of 50° C./10 hPa. Then, ethyl acetate (228 mL, manufactured by FUJIFILM Wako Pure Chemical Corporation) was added thereto and the mixture was stirred at 25° C. for 0.5 hours. The crystals were filtered, washed with diisopropyl ether, and then blast-dried at 40° C. for 12 hours to obtain an intermediate E-25A.

### Synthesis of Intermediate E-25B

Under a nitrogen flow (50 mL/min), the intermediate E-25A (10.0 g, 19.8 mmol), triphenylphosphine (15.6 g, 59.4 mmol, manufactured by FUJIFILM Wako Pure Chemical Corporation), phthalimide (11.0 g, 59.4 mmol, manufactured by Tokyo Chemical Industry Co., Ltd.), and THF (200 mL, manufactured by FUJIFILM Wako Pure Chemical Corporation) were added to a three-neck flask, and the obtained reaction solution was cooled to 0° C.

Next, a dissolving solution obtained by dissolving bis(2-methoxyethyl) azodicarboxylate (DMEAD (registered trademark), 13.9 g, 59.4 mmol, manufactured by FUJIFILM Wako Pure Chemical Corporation) in THF (100 mL) was separately prepared.

While maintaining the internal temperature of the reaction solution obtained above at 5° C. or lower, the dissolving solution was added dropwise to the reaction solution over 2 hours. After completion of the dropwise addition, the reaction solution was stirred at 25° C. for 1 hour.

Next, the reaction solution was cooled to 0° C. and MeOH (300 mL, manufactured by FUJIFILM Wako Pure Chemical Corporation) was added thereto. After the crystal precipitation, the mixture was stirred at 0° C. for 2 hours. The crystals were filtered off, washed with MeOH, and blast-dried at 40° C. for 12 hours to obtain an intermediate E-25B.

### Synthesis of Specific Compound E-25

Under a nitrogen flow (0.1 L/min), the intermediate E-25B (12.0 g, 15.7 mmol) and ethanol (240 mL, manufactured by FUJIFILM Wako Pure Chemical Corporation) were added to a three-neck flask, and the mixture was stirred. Hydrazine monohydrate (1.6 g, 31.4 mmol, manufactured by FUJIFILM Wako Pure Chemical Corporation) was further added to the obtained mixed solution and the mixture was heated under reflux for 1 hour. Thereafter, the obtained reaction solution was cooled to 0° C., and the precipitate was separated by filtration.

MeOH (280 mL) and a 1 M aqueous sodium hydroxide solution (80 mL) were added to the obtained crystal product, and the mixture was stirred at 25° C. for 1 hour. Thereafter, the crystals were separated by filtration, washed with MeOH, and then blast-dried at 40° C. for 12 hours to obtain a specific compound E-25.

The ¹H-NMR data of the obtained specific compound E-25 are shown below.

¹H-NMR (400 MHZ, THF-d8): δ(ppm)=7.04 (d, J=2.0 Hz, 1H), 6.91 (dd, J=2.0 Hz, 8.3 Hz, 1H), 6.85 (d, J=8.3 Hz, 1H), 6.64 (dd, J=10.9 Hz, 17.6 Hz, 1H), 5.61 (dd, J=1.1 Hz, 17.6 Hz, 1H), 5.07 (dd, J=1.1 Hz, J=10.9 Hz, 1H), 4.00 (m, 4H), 2.63 (t, J=6.6 Hz, 4H), 1.48-1.62 (m, 6H), 1.30-1.47 (m, 34H).

### Synthesis of Specific Compound E-30 (Synthesis Example 7)

A specific compound E-30 was synthesized in the same manner as the specific compound E-25, except that 12-bromo-1-dodecanol was changed to 8-bromo-1-octanol in Synthesis Example 6.

The ¹H-NMR data of the obtained specific compound E-30 are shown below.

¹H-NMR (400 MHZ, THF-d8): δ(ppm)=7.04 (d, J=2.0 Hz, 1H), 6.91 (dd, J=2. 0 Hz, 8.3 Hz, 1H), 6.85 (d, J=8.3 Hz, 1H), 6.64 (dd, J=10.9 Hz, 17.6 Hz, 1H), 5.61 (dd, J=1.1 Hz, 17.6 Hz, 1H), 5.07 (dd, J=1.1 Hz, J=10.9 Hz, 1H), 4.0 0 (m, 4H), 2.63 (t, J=6.6 Hz, 4H), 1.48-1.62 (m, 6H), 1.30-1.47 (m, 18H).

### Method for Preparing Composition

The compositions used in Examples and Comparative Examples were prepared by mixing the respective components at the proportions shown in the table which will be given later.

Furthermore, the preparation, filling, storage, and the like of the composition were all performed in a clean room that satisfies a level equal to or lower than ISO Class 2. In addition, the container used for the preparation, filling, storage, and the like of the composition was used after being cleaned with a solvent used for the preparation or the prepared composition.

### Evaluation Method

According to the following procedure, a substrate in which a film consisting of a specific compound was formed was prepared using the composition which will be described later, and the ALD inhibitory properties were evaluated.

### Manufacture of Evaluation Sample Substrate

First, a W layer wafer in which a tungsten layer was formed by a chemical vapor deposition (CVD) method on one surface of a commercially available silicon wafer (diameter: 12 inches) and a Cu layer wafer in which a copper layer was formed by a sputtering method were prepared as a substrate. The film forming conditions were adjusted so that the thicknesses of the W layer and the Cu layer were each 20 nm.

The silicon wafer, and the W layer wafer and the Cu layer wafer obtained by film formation as described above were each cut into a 2 cm square, subjected to a rinsing treatment with isopropyl alcohol (IPA), and then dried by blowing nitrogen gas to each wafer.

The rinsing treatment was carried out by immersing the substrate in IPA, in which the immersion was performed while stirring IPA placed in a container with a magnetic stirrer under the condition of 250 rpm. The temperature of IPA was 25° C. and the immersion time was 30 seconds.

Each wafer after the rinsing treatment was immersed in each composition. Each wafer was immersed in the composition while stirring the composition placed in a container under the condition of 250 rpm using a magnetic stirrer. The temperature of the composition was 25° C. and the immersion time was 10 minutes.

Each wafer after the immersion treatment was subjected to a rinsing treatment with IPA by the same procedure as described above, and then dried with nitrogen gas to obtain a sample (evaluation sample) in which a film obtained by applying the composition was formed on each wafer.

### Evaluation of Contact Angle

Using each evaluation sample obtained by the method, the contact angle of a film obtained by applying the composition to pure water was measured by the following method.

The measurement was carried out in an environment of 23° C. using a fully automatic contact angle meter DMo-901 (manufactured by Kyowa Interface Science Co., Ltd.). The value after 500 milliseconds after the liquid droplet of pure water came into contact with the surface was measured three times, and an average value thereof was defined as the contact angle (deg. (degrees)). Furthermore, the analysis was carried out with a surface tension of pure water set to 72.9 mN/m.

### ALD Inhibitory Properties (Al₂O₃ Deposition Inhibitory Properties and TaN Deposition Inhibitory Properties)

Using an atomic layer deposition device (AD-230LP, manufactured by SAMCO Inc.), an aluminum oxide (Al₂O₃) layer or a tantalum nitride (TaN) layer (ALD coating film) was formed on the sample (evaluation sample) obtained in [Manufacture of Evaluation Sample Substrate] and a sample in which the coating film layer consisting of the specific compound was not formed (untreated sample).

In a case where the Al₂O₃ layer was formed, the ALD treatment temperature was set to 200° C., trimethylaluminum was used as an organic metal raw material, and water was used as an oxidant. In addition, in a case where the TaN layer was formed, the ALD treatment temperature was set to 300° C., pentakis(dimethylamino) tantalum (PDMAT) was used as an organic metal raw material, and ammonia was used as a reducing agent.

Furthermore, each sample (untreated sample) was subjected to the ALD treatment under the condition that the film thickness was 5 nm, in a case where the coating film was not formed of the specific compound.

The film thickness of the ALD coating film in the sample after the ALD treatment was measured using an X-ray fluorescence analysis (XRF) device (AZX⁴⁰⁰ manufactured by Rigaku Corporation). The film thickness of the ALD coating film was measured at five points of the sample and an average value thereof was taken as the film thickness.

The film thickness (nm) of the ALD coating film in the evaluation sample was evaluated according to the following evaluation standard. It means that the smaller the film thickness (nm) of the ALD coating film of the evaluation sample, the more difficult it is for the coating film to be deposited by the ALD treatment, and the better the vapor deposition inhibitory properties of the aluminum oxide layer or the tantalum nitride layer.

### (Evaluation Standard)

S: The film thickness of the ALD coating film is less than 1.0 nm.
A: The film thickness of the ALD coating film is 1.0 nm or more and less than 1.5 nm.
B: The film thickness of the ALD coating film is 1.5 nm or more and less than 2.0 nm.
C: The film thickness of the ALD coating film is 2.0 nm or more and less than 2.5 nm.
D: The film thickness of the ALD coating film is 2.5 nm or more and less than 3.0 nm.
E: The film thickness of the ALD coating film is 3.0 nm or more and less than 3.5 nm.
F: The film thickness of the ALD coating film is 3.5 nm or more.

### Results

Tables 1 to 3 and Tables 4 and 5, which will be described later, show the respective components used in the preparation of the composition and content ratios (mass ratios) thereof. In addition, the evaluation results using the W layer wafer are shown in Tables 1, 2, and 4, and the evaluation results using the Cu layer wafer are shown in Tables 3 and 5. Tables 1 to 3 show the evaluation results of the Al₂O₃ vapor deposition inhibitory properties, and Tables 4 and 5 show the evaluation results of the TaN vapor deposition inhibitory properties.

In the tables, the numerical value indicating the content of each component means the content (the unit is “part by mass”) of each component in a case where the total mass of the respective compositions is 100 parts by mass.

In addition, the “Balance” is an amount adjusted such that the total amount of the components (the specific compound and the polymerization inhibitor) other than the solvent and the solvent included in each composition is 100 parts by mass.

In Tables 1, 2, and 4, the column of “Conjugate acid pKa” indicates a value of an acid dissociation constant of a conjugate acid of the specific compound.

In Tables 3 and 5, the column of “pKa” indicates a value of an acid dissociation constant of the specific compound. Furthermore, as described above, the acid dissociation constant is a value determined by computation from a value based on a Hammett substituent constant and the database of known literature values, using the following software package 1.

Software Package 1: Advanced Chemistry Development (ACD/Labs) Software V 8.14 for Solaris (1994-2007 ACD/Labs)

In Tables 1 to 3 and Tables 4 and 5, the structures or names of the respective components used for the preparation of the composition are shown below.

### Specific Compound

The structures of the specific compounds (E-1 to E-35) are shown below. Furthermore, E-1 to E-13, E-22, and E-23 to E-32 are specific compounds having a basic functional group as the specific functional group, and E-14 to E-21 and E-33 to E-35 are specific compounds having an acidic functional group as the specific functional group.

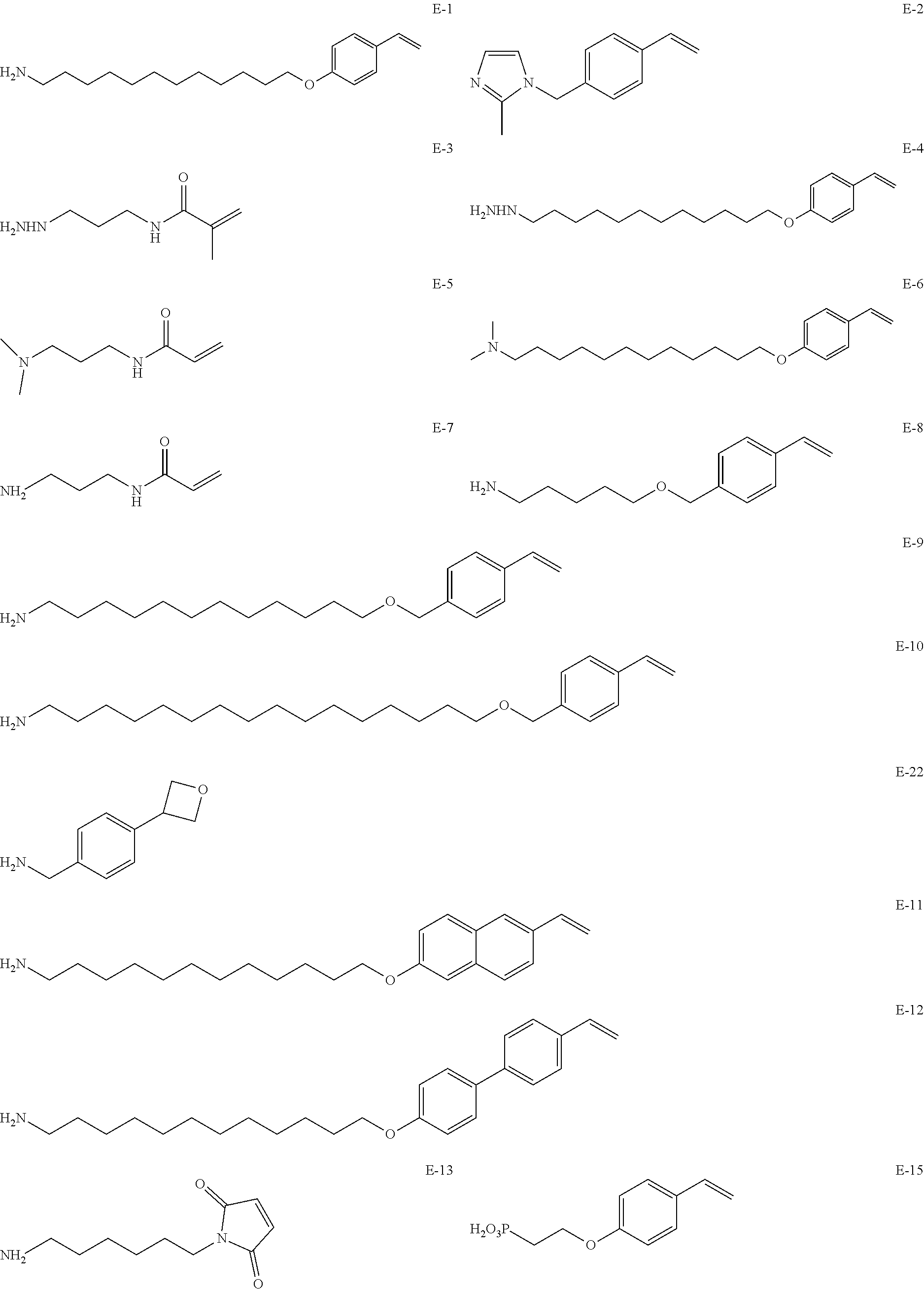

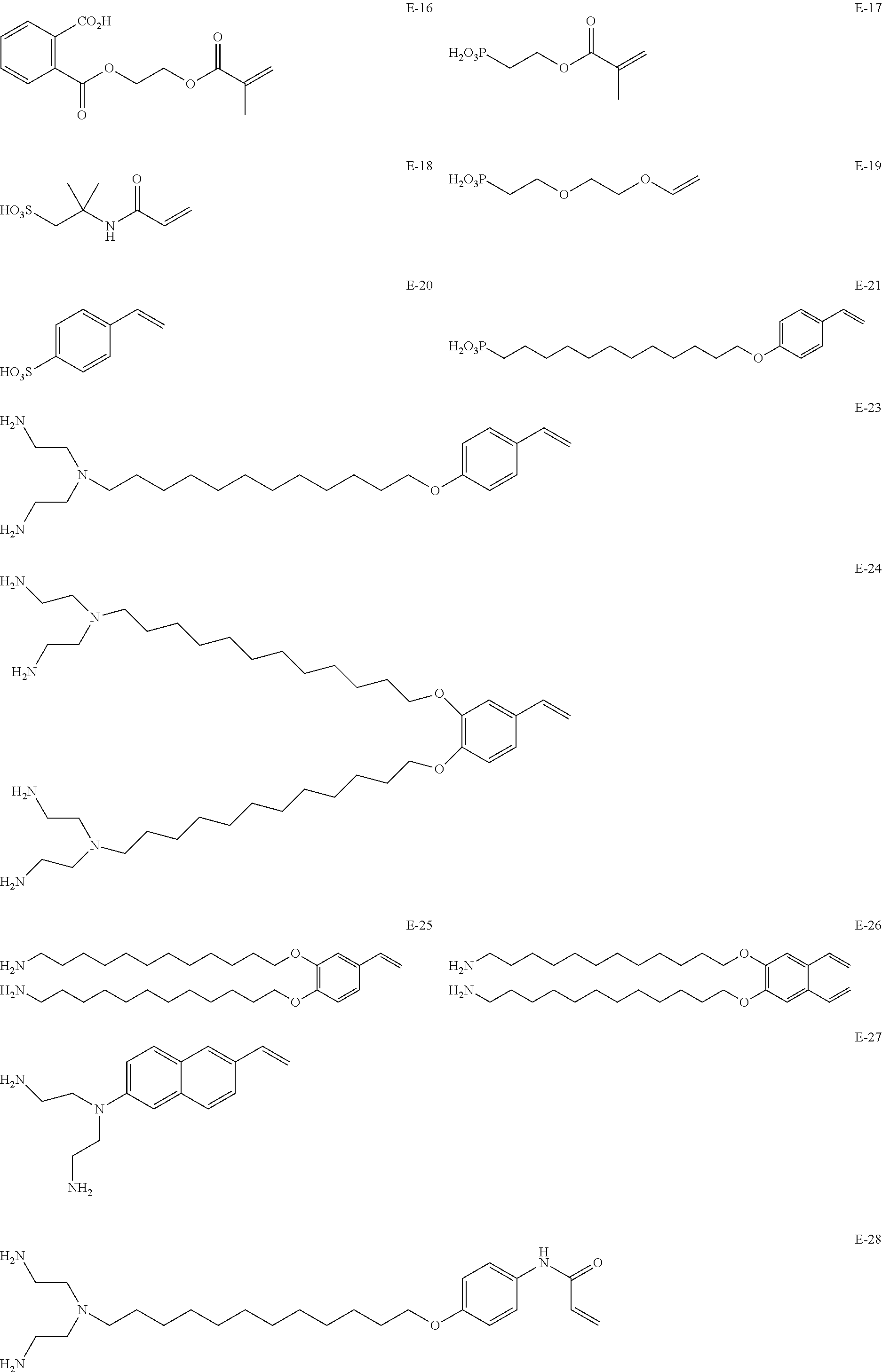

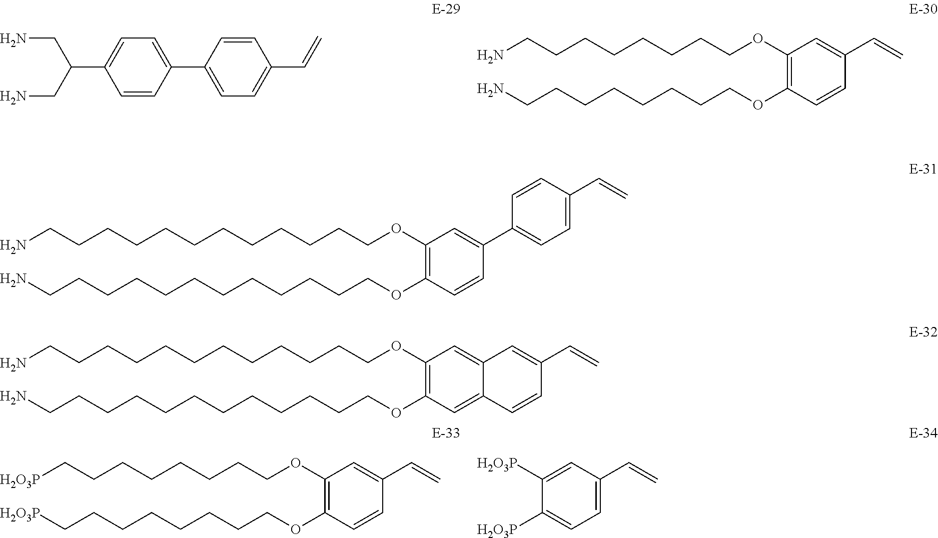

### Comparative Compound

The structures of comparative compounds (CE-1 and CE-2) used for comparison with the specific compound are shown below.

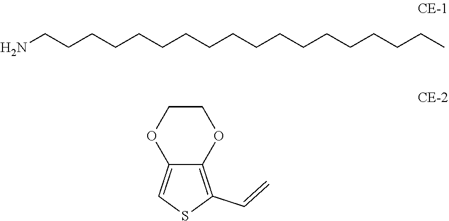 [SMILES: CCCCCCCCCCCCCCCCCCN ; C=Cc1scc2c1OCCO2]

### Solvent

PGME: Propylene glycol monomethyl ether
EtOH: Ethanol.
IPA: 2-Propanol

### Polymerization Inhibitor

A-1: 4-Methoxyphenol
A-2: 1,4-Benzoquinone
A-3: Phenothiazine
A-4: 4-Hydroxy-2,2,6,6-tetramethylpiperidine 1-oxyl
A-5: 2,2-Diphenyl-1-picrylhydrazyl
A-6: 2,2,6,6-Tetramethylpiperidine 1-oxyl

TABLE 1

| | | | Example | Example | Example | Example | Example | Example | Example | Example | Example |
|---|---|---|---|---|---|---|---|---|---|---|---|
| | | | A1 | A2 | A3 | A4 | A5 | A6 | A7 | A8 | A9 |
| Composition | Specific | Type | E-1 | E-2 | E-3 | E-4 | E-5 | E-6 | E-7 | E-8 | E-9 |
| | compound | Amount | 0.10 | 0.10 | 0.10 | 0.10 | 0.10 | 0.10 | 0.10 | 0.10 | 0.10 |
| | | (parts | | | | | | | | | |
| | | by mass) | | | | | | | | | |
| | | Conjugate | 10.7 | 7.0 | 8.2 | 8.7 | 9.4 | 9.8 | 9.7 | 10.7 | 10.7 |
| | | acid | | | | | | | | | |
| | | pKa | | | | | | | | | |
| | | Type | — | — | — | — | — | — | — | — | E-1 |
| | | Amount | — | — | — | — | — | — | — | — | 0.10 |
| | | (parts | | | | | | | | | |
| | | by mass) | | | | | | | | | |
| | | Conjugate | — | — | — | — | — | — | — | — | 10.7 |
| | | acid | | | | | | | | | |
| | | pKa | | | | | | | | | |
| | Polymer- | Type | — | — | — | — | — | — | — | — | — |
| | ization | Amount | — | — | — | — | — | — | — | — | — |
| | inhibitor | (parts | | | | | | | | | |
| | | by mass) | | | | | | | | | |
| | Solvent | Type | PGME | PGME | PGME | PGME | PGME | PGME | PGME | PGME | PGME |
| | | Amount | Balance | Balance | Balance | Balance | Balance | Balance | Balance | Balance | Balance |
| | | (parts | | | | | | | | | |
| | | by mass) | | | | | | | | | |
| | Total | | 100 | 100 | 100 | 100 | 100 | 100 | 100 | 100 | 100 |
| | (parts by mass) | | | | | | | | | | |
| Evaluation | Pure water | W | 75 | 65 | 50 | 70 | 48 | 73 | 48 | 60 | 75 |
| | contact angle | | | | | | | | | | |
| | after substrate | | | | | | | | | | |
| | modification | | | | | | | | | | |
| | (deg.) | | | | | | | | | | |
| | Al₂O₃ | W | S | E | D | C | C | B | A | S | S |
| | Deposition | | | | | | | | | | |
| | inhibitory | | | | | | | | | | |
| | properties | | | | | | | | | | |

TABLE 2

| | | | Example | Example | Example | Example | Example | Example | Example |
|---|---|---|---|---|---|---|---|---|---|
| | | | A10 | A11 | A12 | A13 | A14 | A15 | A16 |
| Compo- | Specific | Type | E-10 | E-11 | E-12 | E-13 | E-1 | E-1 | E-1 |
| sition | compound | Amount | 0.10 | 0.10 | 0.10 | 0.10 | 3.00 | 0.001 | 0.01 |
| | | (parts | | | | | | | |
| | | by | | | | | | | |
| | | mass) | | | | | | | |
| | | Conjugate | 10.7 | 10.7 | 10.7 | 10.7 | 10.7 | 10.7 | 10.7 |
| | | acid pKa | | | | | | | |
| | | Type | — | — | — | — | — | — | — |
| | | Amount | — | — | — | — | — | — | — |
| | | (parts | | | | | | | |
| | | by | | | | | | | |
| | | mass) | | | | | | | |
| | | Conjugate | — | — | — | — | — | — | — |
| | | acid pKa | | | | | | | |
| | Polymer- | Type | A-1 | A-2 | A-3 | — | — | — | — |
| | ization | Amount | 0.00001 | 0.00001 | 0.00001 | — | — | — | — |
| | inhibitor | (parts | | | | | | | |
| | | by | | | | | | | |
| | | mass) | | | | | | | |
| | Solvent | Type | PGME | PGME | PGME | PGME | PGME | PGME | PGME |
| | | Amount | Balance | Balance | Balance | Balance | Balance | Balance | Balance |
| | | (parts | | | | | | | |
| | | by | | | | | | | |
| | | mass) | | | | | | | |
| | Total (parts by mass) | | 100 | 100 | 100 | 100 | 100 | 100 | 100 |
| Eval- | Pure water | W | 80 | 81 | 82 | 50 | 77 | 70 | 75 |
| uation | contact | | | | | | | | |
| | angle | | | | | | | | |
| | after | | | | | | | | |
| | substrate | | | | | | | | |
| | modification | | | | | | | | |
| | (deg.) | | | | | | | | |
| | Al₂O₃ | W | S | S | S | A | S | A | A |
| | Deposition | | | | | | | | |
| | inhibitory | | | | | | | | |
| | properties | | | | | | | | |

| | | | Example | Comparative | Comparative | Comparative |
|---|---|---|---|---|---|---|
| | | | A17 | Example CA1 | Example CA2 | Example CA3 |
| Compo- | Specific | Type | E-22 | — | CE-1 | CE-2 |
| sition | compound | Amount | 0.10 | — | 0.10 | 0.10 |
| | | (parts | | | | |
| | | by | | | | |
| | | mass) | | | | |
| | | Conjugate | 9.4 | — | 10.7 | — |
| | | acid pKa | | | | |
| | | Type | — | — | — | — |
| | | Amount | — | — | — | — |
| | | (parts | | | | |
| | | by | | | | |
| | | mass) | | | | |
| | | Conjugate | — | — | — | — |
| | | acid pKa | | | | |
| | Polymer- | Type | — | — | — | — |
| | ization | Amount | — | — | — | — |
| | inhibitor | (parts | | | | |
| | | by | | | | |
| | | mass) | | | | |
| | Solvent | Type | PGME | PGME | PGME | PGME |
| | | Amount | Balance | Balance | Balance | Balance |
| | | (parts | | | | |
| | | by | | | | |
| | | mass) | | | | |
| | Total (parts by mass) | | 100 | 100 | 100 | 100 |
| Eval- | Pure water | W | 70 | 30 | 104 | 30 |
| uation | contact | | | | | |
| | angle | | | | | |
| | after | | | | | |
| | substrate | | | | | |
| | modification | | | | | |
| | (deg.) | | | | | |
| | Al₂O₃ | W | B | F | F | F |
| | Deposition | | | | | |
| | inhibitory | | | | | |
| | properties | | | | | |

TABLE 3

| | | | | | | | | | | | Compar- |
|---|---|---|---|---|---|---|---|---|---|---|---|
| | | | Ex- | Ex- | Ex- | Ex- | Ex- | Ex- | Ex- | Ex- | ative |
| | | | ample | ample | ample | ample | ample | ample | ample | ample | Example |
| | | | B1 | B2 | B3 | B4 | B5 | B6 | B7 | B8 | CB1 |
| Composition | Specific compound | Type | E-14 | E-15 | E-16 | E-17 | E-18 | E-19 | E-20 | E-21 | — |
| | | Amount | 0.10 | 0.10 | 0.10 | 0.10 | 0.10 | 0.10 | 0.10 | 0.10 | — |
| | | (parts | | | | | | | | | |
| | | by mass) | | | | | | | | | |
| | | pKa | 2.1 | 4.8 | 3.3 | 1.9 | 1.7 | 2.1 | −0.6 | 2.4 | — |
| | | Type | — | — | — | — | — | — | — | — | — |
| | | Amount | — | — | — | — | — | — | — | — | — |
| | | (parts | | | | | | | | | |
| | | by mass) | | | | | | | | | |
| | | pKa | — | — | — | — | — | — | — | — | — |
| | Polymerization | Type | — | — | — | — | — | — | A-4 | A-5 | — |
| | inhibitor | Amount | — | — | — | — | — | — | 0.00001 | 0.00001 | — |
| | | (parts | | | | | | | | | |
| | | by mass) | | | | | | | | | |
| | Solvent | Type | PGME | PGME | PGME | PGME | PGME | PGME | PGME | PGME | PGME |
| | | Amount | Balance | Balance | Balance | Balance | Balance | Balance | Balance | Balance | Balance |
| | | (parts | | | | | | | | | |
| | | by mass) | | | | | | | | | |
| | Total | | 100 | 100 | 100 | 100 | 100 | 100 | 100 | 100 | 100 |
| | (parts by mass) | | | | | | | | | | |
| Evaluation | Pure water | Cu | 70 | 65 | 55 | 70 | 55 | 73 | 56 | 60 | 50 |
| | contact angle | | | | | | | | | | |
| | after | | | | | | | | | | |
| | substrate | | | | | | | | | | |
| | modification | | | | | | | | | | |
| | (deg.) | | | | | | | | | | |
| | Al₂O₃ | Cu | S | D | D | B | B | B | S | S | F |
| | Deposition | | | | | | | | | | |
| | inhibitory | | | | | | | | | | |
| | properties | | | | | | | | | | |

TABLE 4

| | | | Ex- | Ex- | Ex- | Ex- | Ex- | Ex- | Ex- | Ex- | Ex- | Ex- |
|---|---|---|---|---|---|---|---|---|---|---|---|---|
| | | | ample | ample | ample | ample | ample | ample | ample | ample | ample | ample |
| | | | C1 | C2 | C3 | C4 | C5 | C6 | C7 | C8 | C9 | C10 |
| Composition | Specific | Type | E-23 | E-24 | E-25 | E-26 | E-27 | E-28 | E-29 | E-30 | E-31 | E-32 |
| | compound | Amount | 0.10 | 0.10 | 0.10 | 0.10 | 0.10 | 0.10 | 0.10 | 0.10 | 0.10 | 0.10 |
| | | (parts | | | | | | | | | | |
| | | by mass) | | | | | | | | | | |
| | | Conjugate | 9.9 | 9.9 | 11.0 | 11.0 | 9.6 | 9.9 | 9.7 | 10.7 | 11.0 | 11.0 |
| | | acid | | | | | | | | | | |
| | | pKa | | | | | | | | | | |
| | Polymerization | Type | — | — | — | A-1 | — | A-6 | — | — | — | — |
| | inhibitor | Amount | — | — | — | 0.0001 | — | 0.00001 | — | — | — | — |
| | | (parts | | | | | | | | | | |
| | | by mass) | | | | | | | | | | |
| | Solvent | Type | PGME | PGME | PGME | EtOH | PGME | PGME | IPA | PGME | PGME | PGME |
| | | Amount | Balance | Balance | Balance | Balance | Balance | Balance | Balance | Balance | Balance | Balance |
| | | (parts | | | | | | | | | | |
| | | by mass) | | | | | | | | | | |
| | Total | | 100 | 100 | 100 | 100 | 100 | 100 | 100 | 100 | 100 | 100 |
| | (parts by mass) | | | | | | | | | | | |
| Evaluation | Pure water contact | W | 73 | 70 | 60 | 75 | 74 | 56 | 61 | 60 | 60 | 60 |
| | angle after substrate | | | | | | | | | | | |
| | modification | | | | | | | | | | | |
| | (deg.) | | | | | | | | | | | |
| | TaN | W | A | A | S | S | A | B | A | S | S | S |
| | Deposition | | | | | | | | | | | |
| | inhibitory | | | | | | | | | | | |
| | properties | | | | | | | | | | | |

TABLE 5

| | | | Example | Example | Example |
|---|---|---|---|---|---|
| | | | D1 | D2 | D3 |
| Composition | Specific | Type | E-33 | E-34 | E-35 |
| | compound | Amount | 0.10 | 0.10 | 0.10 |
| | | (parts by mass) | | | |
| | | pKa | 2.4 | 1.4 | 1.8 |
| | Polymerization | Type | — | — | — |
| | inhibitor | Amount | — | — | — |
| | | (parts by mass) | | | |
| | Solvent | Type | PGME | PGME | PGME |
| | | Amount | Balance | Balance | Balance |
| | | (parts by mass) | | | |
| | Total (parts by mass) | | 100 | 100 | 100 |
| Evaluation | Pure water contact angle | Cu | 61 | 70 | 70 |
| | after substrate modification | | | | |
| | (deg.) | | | | |
| | TaN | Cu | S | A | S |
| | Deposition inhibitory properties | | | | |

From the results of Tables 1 to 3 and Tables 4 and 5, it was confirmed that the composition of the embodiment of the present invention can form a coating film having high ALD inhibitory properties for formation of a coating film.

On the other hand, since the compositions of Comparative Examples include a compound having only one of a polymerizable group or a specific functional group and do not include the specific compound, a sufficient effect of inhibitory properties for formation of an ALD coating film was not obtained.

In addition, from the comparison between Example A2 and Example A4, and the like, it was confirmed that in a case where the specific functional group in the specific compound is an amino group, a hydrazine group, or a guanidine group, the effect of the present invention is more excellent.

From the comparison between Example A3 and Example A5, and the like, it was confirmed that in a case where the specific functional group in the specific compound is a primary amino group, a secondary amino group, or a tertiary amino group, the effect of the present invention is more excellent.

From the comparison between Example A5 and Example A7, and the like, it was confirmed that in a case where the specific functional group in the specific compound is a primary amino group, the effect of the present invention is more excellent.

From the comparison between Examples A7 and A13 and Example A17, and the like, it was confirmed that in a case where the polymerizable group in the specific compound is an aromatic vinyl group, an acryloyloxy group, a methacryloyloxy group, an acrylamide group, a methacrylamide group, a maleimide group, or a vinyl ether group, the effect of the present invention is more excellent.

From the comparison between Example A5 and Example A6, and the like, it was confirmed that in a case where the polymerizable group in the specific compound is a styryl group or a vinylnaphthyl group, the effect of the present invention is more excellent.

From the comparison between Examples Al and A8 to A12 and Examples A7 and A13, it was confirmed that in a case where the specific compound is a compound represented by General Formula (1), the effect of the present invention is more excellent.

From the comparison of Examples B2 to B6, and the like, it was confirmed that in a case where the specific functional group in the specific compound is a phosphonic acid group or a sulfo group, the effect of the present invention is more excellent.

From the comparison between Examples B1 and B7 to B8 and Examples B4 to B6, and the like, it was confirmed that in a case where the specific compound is the compound represented by General Formula (1), the effect of the present invention is more excellent.

In addition, from the comparison of Examples C1 to C10, the comparison of Examples D1 to D3, and the like, it was confirmed that in a case where the specific compound is a compound represented by General Formula (4), the effect of the present invention is more excellent.

In addition, it was confirmed that in a case where the ALD inhibitory properties were evaluated under the same conditions as in Example A4, except that 0.095 parts by mass of the specific compound E-4 and 0.005 parts by mass of the comparative compound CE-1 in combination were used instead of using 0.10 parts by mass of the specific compound E-4 in Example A4 in Table 1, the same effect as in Example A4 could be obtained.

What is claimed is:

1. A composition for treating a semiconductor device, the composition comprising:
a compound having a specific functional group bonded to or adsorbed on a substrate and a polymerizable group; and
a solvent,
wherein the specific functional group is a basic functional group or an acidic functional group,
in a case where the specific functional group is the basic functional group, an acid dissociation constant of a conjugate acid of a compound obtained by adding a proton to the basic functional group is 7.0 or more, and
in a case where the specific functional group is the acidic functional group, an acid dissociation constant of the compound upon dissociation of a proton from the acidic functional group is 5.0 or less.

2. The composition for treating a semiconductor device according to claim 1,
wherein the basic functional group is an amino group, a hydrazine group, or a guanidine group.

3. The composition for treating a semiconductor device according to claim 2,
wherein the basic functional group is a primary amino group, a secondary amino group, or a tertiary amino group.

4. The composition for treating a semiconductor device according to claim 3,
wherein the basic functional group is a primary amino group.

5. The composition for treating a semiconductor device according to claim 1,
wherein the acidic functional group is a phosphonic acid group, a sulfo group, or a carboxy group.

6. The composition for treating a semiconductor device according to claim 5,
wherein the acidic functional group is a phosphonic acid group or a sulfo group.

7. The composition for treating a semiconductor device according to claim 1,
wherein the polymerizable group is an ethylenically unsaturated group.

8. The composition for treating a semiconductor device according to claim 7,
wherein the polymerizable group is an aromatic vinyl group, an acryloyloxy group, a methacryloyloxy group, an acrylamide group, a methacrylamide group, a maleimide group, or a vinyl ether group.

9. The composition for treating a semiconductor device according to claim 1,
wherein the polymerizable group is a styryl group or a vinylnaphthyl group.

10. The composition for treating a semiconductor device according to claim 1,
wherein the compound is a compound represented by General Formula (1), 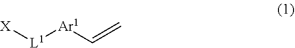
in General Formula (1),
X represents a primary amino group, a phosphonic acid group, or a sulfo group,
L¹ represents a single bond or an alkylene group which may have an etheric oxygen atom, and
Ar¹ represents an arylene group.

11. The composition for treating a semiconductor device according to claim 1,
wherein the compound is a compound represented by General Formula (4), 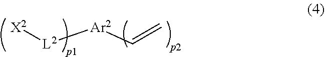
in General Formula (4),
X² represents a primary amino group, a phosphonic acid group, or a sulfo group,
L² represents an alkylene group which may have an etheric oxygen atom,
p1 represents an integer of 2 or more,
p2 represents 1 or 2, and
Ar² represents a (p1+p2)-valent aromatic ring group.

12. The composition for treating a semiconductor device according to claim 1,
wherein a content of the compound is 3.00% by mass or less with respect to a total mass of the composition for treating a semiconductor device.

13. The composition for treating a semiconductor device according to claim 1,
wherein a total amount of the compound and the solvent is 99.90% by mass or more with respect to a total mass of the composition for treating a semiconductor device.

14. The composition for treating a semiconductor device according to claim 1,
wherein a water contact angle of a film obtained by applying the composition for treating a semiconductor device is 60 degrees or more.

15. A compound represented by General Formula (2), 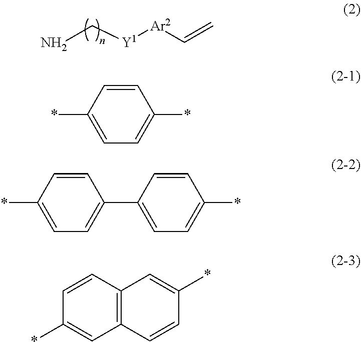
in General Formula (2),
Y¹ represents —O— or —OCH₂—,
Ar² represents a group represented by any of Formulae (2-1) to (2-3),
n represents an integer of 5 or more, and
* represents a bonding position.

16. A compound represented by General Formula (3), 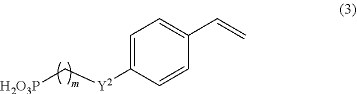
in General Formula (3),
Y² represents —O— or —OCH₂—, and
m represents an integer of 2 or more.

17. A compound represented by General Formula (5), 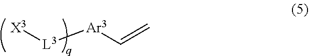
in General Formula (5),
X³ represents a primary amino group or a phosphonic acid group,
L³ represents —(CH₂)ᵣO— or an alkylene group having 6 to 18 carbon atoms,
r represents an integer of 2 to 18,
Ar³ represents a group obtained by removing (q+1) pieces of hydrogen atoms from an aromatic hydrocarbon selected from the group consisting of benzene, biphenyl, and naphthalene, and
q represents an integer of 2 or more.

18. A method for producing a modified substrate, the method comprising:
a step 1 of bringing a substrate having at least two surfaces of a first surface and a second surface, which are composed of materials different from each other, into contact with the composition for treating a semiconductor device according to claim 1, to form a first film on the first surface; and
a step 2 of subjecting the substrate obtained in the step 1 to an atomic layer deposition treatment (ALD treatment) to form a second film on the second surface.

19. A method for producing a semiconductor device, the method comprising:
the method for producing a modified substrate according to claim 18.

20. The composition for treating a semiconductor device according to claim 1,
wherein the basic functional group is a primary amino group, a secondary amino group, or a tertiary amino group.
